(19) United States
(12) Patent Application Publication
(10) Pub. No.: US 2002/0164348 A1
(43) Pub. Date: Nov. 7, 2002

(54) Inhibitors of viral infection

(76) Inventors: Karla Kirkegaard, Palo Alto, CA (US); Kathy Richmond, Mountain View, CA (US); John Lyle, Palo Alto, CA (US); Steve Schultz, Tsaile, AZ (US); Scott Hobson, Schriesheim (DE)

Correspondence Address: Carol L. Francis, BOZICEVIC, FIELD & FRANCIS LLP, Suite 200, 200 Middlefield Road, Menlo Park, CA, 94025, US

(21) Appl. No.: 09/863,841

(22) Filed: May 22, 2001

Related U.S. Application Data
(60) Provisional application No. 60/206,887, filed on May 24, 2000.

(51) Int. Cl. A61K 39/12; C12Q 1/70

(52) U.S. Cl. 424/186.1; 530/388.3; 435/5

(57) ABSTRACT

The present invention provides a viral RNA-dependent RNA polymerase pharmacophore which is characterized by binding to a conserved interface binding surfaces of a viral RNA-dependent RNA polymerase.

### CROSS-REFERENCE TO RELATED APPLICATION

This application claims the benefit of earlier filed U.S. provisional application serial No. 60/206,887, filed May 24, 2000, which application is incorporated herein by reference in its entirety.

### GOVERNMENT RIGHTS

The United States Government may have certain rights in this application pursuant to National Institutes of Health Grant 42119, and DARPA Grant Number N65236-98-1-5405.

### FIELD OF THE INVENTION

This invention relates generally to viral RNA polymerases, functional pharmacophores of these polymerases, and methods of inhibit viral replication. More specifically, this invention relates to conserved binding surfaces of viral RNA-dependent RNA polymerases and to pharmacophores which mimic and/or bind to these binding surfaces.

### BACKGROUND OF THE INVENTION

Especially rapid evolution is a hallmark of RNA viruses due to the high error rates of RNA-dependent RNA polymerases. These polymerases are estimated to introduce errors at rates of 1 in 1000 to 10,000 nucleotides polymerized, and thus a great deal of variation exists in every population of RNA viruses. Any selective pressure, such as the development of an immune response in an infected host or treatments with drugs, can result in the rapid outgrowth of any viral variants that escape the inhibitory pressure. Over time, this has resulted in the bewildering array of RNA viruses that currently infect human and other populations, including hundreds of strains of the common cold caused by rhinoviruses, dozens of influenza virus serotypes, and at least 70 different flaviviruses, some of which cause lethal hemorrhagic fevers.

Positive-strand RNA viruses all follow a similar replicative strategy and, indeed, there is much conservation in the biochemical properties of the proteins required for intracellular amplification of the viral RNA genomes. Within a cell, the incoming positive-strand RNA is sufficient to encode all the proteins needed to initiate the infectious cycle, which will require synthesis of negative-strand and positive-strand RNAs by the viral RNA-dependent RNA polymerase. After entry into host cells, incoming RNA genomes are translated by cellular ribosomes. Most RNA viruses encode polyproteins, which are then cleaved by viral proteases into the individual viral proteins. One of the most conserved of these proteins is an RNA-dependent RNA polymerase, needed by all positive-strand RNA viruses to copy their positive-strand genome into a complementary negative strand, and then to copy the negative strand to churn out large amounts of the positive-strand genome. Other proteins whose functions appear conserved among positive-strand RNA viruses are membrane-associated proteins that are also found in the RNA replication complexes, sometimes by virtue of direct association with the RNA-dependent RNA polymerase (Grun, J. B. and Brinton M. A. J. Virol. 61:3641-3644 (1987); Hope, D. A et al., J. Virol. 71:9490-8 (1997); Kao, C. Cet al. J. Virol. 66: 6322-6329 (1992)).

Poliovirus RNA-dependent RNA polymerase, for example, binds to its RNA substrates highly cooperatively, with multiple polymerase molecules binding to each molecule of substrate RNA. Such cooperative RNA binding was also required for RNA polymerase activity in solution (Pata, J. D. et al., RNA,. 1:466-477(1995)). Recently, the three-dimensional structure of poliovirus type 1 Mahoney was solved by X-ray crystallography (Hansen, J. L. et al., Structure 5:1109-1122 (1997). While showing many similarities to other polymerases, especially to the RNA-dependent DNA polymerase (reverse transcriptase) from HIV type 1, the structure of the poliovirus RNA-dependent RNA polymerase showed many surprising and exciting features (Id.).

The RNA-dependent RNA polymerases offer attractive targets for therapeutic intervention of multiple viral diseases. There is thus a need in the art for agents that inhibit viral infection and/or disease progression by inhibiting activity of these polymerases.

### SUMMARY OF THE INVENTION

The present invention provides a pharmacophore characterized by binding to a viral RNA polymerase, and in particular an interface binding surface on an RNA-dependent RNA polymerase. The pharmacophore is can be designed to bind specifically to a particular virus or strain, e.g., poliovirus, or can be designed to bind to multiple different viral polymerase having the conserved interface binding surfaces. The pharmacophores of the present invention selectively bind to one or more binding surfaces which are defined in terms of their amino acid sequence and structural aspects. In particular, two binding surfaces involved in polymerase oligomerization, Interface I and Interface II of RNA-dependent RNA polymerases, are described as preferred binding surfaces of the pharmacophores of the invention. Using the 3D coordinates defining the interface binding surfaces of viral RNA-dependent RNA polymerases, functionally equivalent pharmacophores are disclosed and described as are methods of generating such pharmacophores. These pharmacophores are useful in assays and as therapeutic agents to prevent and/or treat viral infection.

An aspect of the invention is the 3-dimensionally defined interface binding surfaces of the viral polymerases and functionally equivalent pharmacophores of these surfaces.

Another aspect of the invention is pharmacophores with a 3-dimensionally defined surface in the negative image of one or more binding surfaces of a viral RNA-dependent RNA polymerase.

Yet another aspect of the invention comprises administering a viral pharmacophore of the invention to a subject infected with a virus which expresses RNA polymerase having the interface binding surfaces as described.

Yet another aspect of the invention are the binding sites of picornaviral polymerase-polymerase interaction, which are defined by the negative image of the defined 3-dimensional space of the described interface binding surfaces.

It is one object of the present invention to develop antiviral compounds that will target a large segment of RNA viruses, i.e. the positive-strand RNA viruses.

Another object of the invention is to provide an isolated protein or peptide pharmacophore which is characterized by binding to a viral RNA-dependent RNA polymerase and interruption of polymerase-polymerase interaction.

Yet another object is to provide antibodies which bind to a viral RNA-dependent RNA polymerase and disrupt polymerase-polymerase interaction.

Yet another object of the invention is to provide a small molecule pharmacophore which is characterized by binding to a viral RNA-dependent RNA polymerase and interruption of polymerase-polymerase interaction.

An advantage of the invention is that the pharmacophores of the invention can be used to detect the presence of a viral RNA-dependent RNA polymerase in a subject.

Another advantage of the invention is that the pharmacophores affect polymerase oligomerization, and thus the therapeutic effect of these pharmacophores is not easily circumvented through selective pressure.

These and other objects, advantages, and features of the invention will become apparent to those persons skilled in the art upon reading the details of the interface binding surfaces, design of the pharmacophores, and inhibition of the viral progression as more fully described below.

### BRIEF DESCRIPTION OF THE DRAWINGS

FIG. 1 is a ribbon diagram of the structure of the poliovirus polymerase, illustrating the α-carbon backbone.

FIGS. 2A, 2B and 2C illustrate the specific interface interactions between poliovirus polymerase molecules seen as in the crystal structure.

FIG. 3 illustrates the rationale for choosing oligomeric protein interaction surfaces as excellent targets for drug discovery for RNA viruses. Circles represent proteins encoded by linear RNA genomes; a mutation in an RNA genome is denoted by an X; and the resulting mutant protein by a filled circle. Proteins whose function is destroyed in the presence of an inhibitory drug are crossed out in the bottom half of the figure.

FIG. 4 is a graph illustrating binding of the WT (wildtype) and the Interface I mutant polymerases to duplex 0.20 RNA as a function of polymerase concentration.

FIG. 5 is a graph demonstrating that polymerases containing mutations at Interface I incorporate nucleotides into a dT₁₆-primed poly-A substrate to a lesser extent than WT polymerase.

FIG. 6 is a ribbon drawing of an intermolecular interaction via Interface II.

FIG. 7 is a graph illustrating RNA binding by the N-terminal deletion mutants using the 5.20 RNA.

FIG. 8 is a graph illustrating the ability of the N-terminal deletion mutants and the point mutants in the N-terminal interaction region to incorporate nucleotides into a dT₁₆-primed poly-A substrate,

FIG. 9 is a graph illustrating the specific activity of poliovirus polymerase at various concentrations under conditions in which substrate is in excess and enzyme is limiting.

FIG. 10 illustrates the ability of Peptide 1 to inhibit poliovirus polymerase activity.

FIGS. 11 and 12 illustrate the effect of Peptide 1 on polymerase incorporation of a homopolymeric template with and without limiting primer, respectively.

FIG. 13 is a series of electron microscopy results illustrating the ability of Peptide 1 to inhibit the wild-type polymerase lattice formation.

FIG. 14 is a graph showing the results of homologous and heterologous RNA-dependent RNA polymerase interactions in a yeast two hybrid system. This data shows that heterologous polymerase interactions can form between polymerases from viruses of different families, such as picornaviruses and alphaviruses. Polio 3D=poliovirus 3D; HRV 3D=human rhinovirus 14 3D; Ent71 3D=enterovirus 71 3D; HAV 3D=hepatitis A 3D; Sin ns P4=sinbdbis virus nsP4; Lex202=Lex202 vector control.

FIG. 15 is a graph showing the results of yeast two hybrid experiments to examine formation of Interface II interactions between heterologous polymers using N-terminal sequences of polymerases from rhinovirus 14 (IntII HRV14), rhinovirus 2 (IntII HRV12) and rhinovirus 16 (IntII HRV16) as bait, and with poliovirus polymerase (D65-PJG) serving as prey. These data show that the N-terminal residues of three different serotypes of rhinovirus can form an intermolecular interaction with the heterologous polymerase from poliovirus at interface ii.

### DETAILED DESCRIPTION OF PREFERRED EMBODIMENTS

Before the present peptides, polymerases, and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a peptide” includes a plurality of such peptides and reference to “the virus” includes reference to one or more virus and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

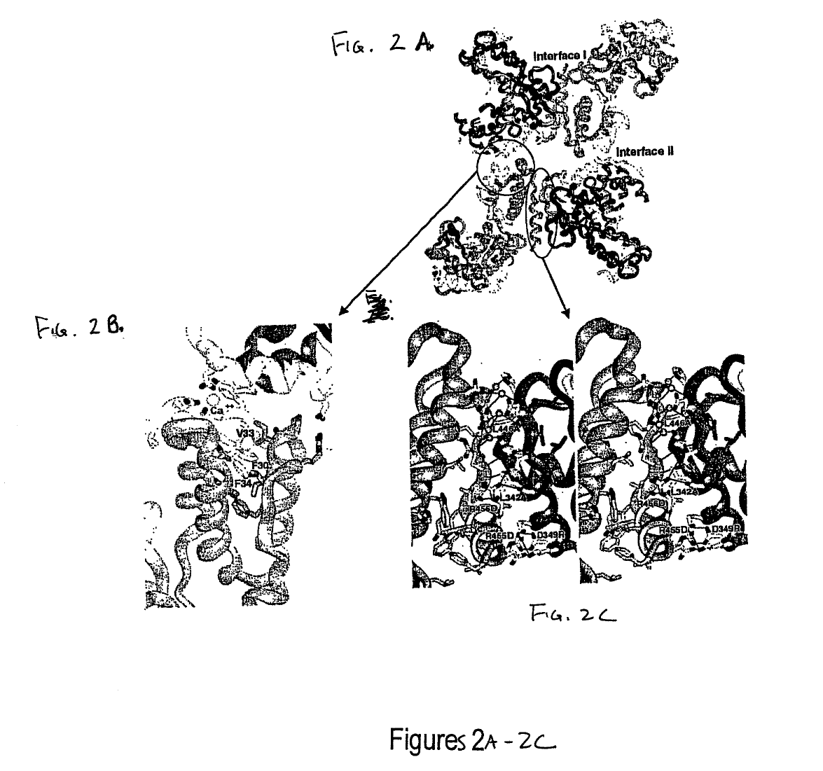

A pharmacophore is a compound that has a specific biochemical activity which is obtained by the 3-dimensional physical shape of the compound and the electrochemical properties of the atoms making up the compound. Specific pharmacophores of the invention are defined by the structural aspects of the conserved binding surfaces Interface I, Interface II, or both of a viral RNA-dependent RNA polymerase. Thus a pharmacophore of the invention can have a shape (i.e., the geometric specifications) substantially as defined by the structure of FIG. 2A, and more specifically as defined by the Interface regions of FIGS. 2B and 2C or a negative image thereof. The term “pharmacophore” is meant to encompass synthetic, naturally occurring, or recombinantly produced molecules (e.g., small molecule; drugs; peptides; antibodies (including antigen-binding antibody fragments, e.g., to provide for passive immunity); endogenous factors present in eukaryotic or prokaryotic cells (e.g., polypeptides, plant extracts, and the like); etc.). Of particular interest are screening assays for agents that have a low toxicity for human cells.

Pharmacophores encompass numerous chemical classes, including organic molecules, such as small organic compounds having a molecular weight of more than 50 and less than about 2,500 daltons. Pharmacophores comprise functional groups necessary for structural interaction with proteins, particularly hydrogen bonding, and typically include at least an amine, carbonyl, hydroxyl or carboxyl group, preferably at least two of the functional chemical groups. The pharmacophores often comprise cyclical carbon or heterocyclic structures and/or aromatic or polyaromatic structures substituted with one or more of the above functional groups. Pharmacophores are also found among biomolecules including, but not limited to: peptides, saccharides, fatty acids, steroids, purine and/or pyrimidine containing structures, derivatives, structural analogs or combinations thereof.

Pharmacophores can be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

The terms “treatment”, “treating”, “treat” and the like are used herein to generally mean obtaining a desired pharmacological and/or physiological effect. The effect may be prophylactic in terms of completely or partially preventing a viral infection or progression of a viral infection and/or may be therapeutic in terms of partially or completely curing a viral disease or adverse effect attributable to the disease. The “treatment” as used herein covers any treatment of a disease in a mammal, particularly a human, and includes:

(a) preventing viral infection or symptoms from occurring in a subject which may be infected with the virus but has not yet been diagnosed as having a viral disease;

(b) inhibiting viral disease progression and/or disease symptoms, i.e., arresting the development of the viral disease; or

(c) relieving a viral disease symptom, i.e., causing regression of viral infection or viral disease symptoms.

The term “corresponding position” means the position of an amino acid in a peptide or the position of a codon in a nucleotide sequence corresponds to the same position in the sequence of the conserved binding surface of different viral polymerases, i.e. different but related positive strand virus such as a picornavirus and a flavivirus. Thus a residue in a specific position in Interface I of a poliovirus will have a “corresponding position” in the conserved interface of a different picornavirus, a flavivirus, etc.

### GENERAL ASPECTS OF THE INVENTION

The present invention is based on the finding that particular binding surfaces on viral RNA-dependent RNA polymerases are critical for polymerase-polymerase interactions, for oligomerization of the polymerases, and for RNA polymerase activity in vivo. In a particular embodiment, the binding surface is one of two defined interfaces, Interface I and Interface II, which are involved in polymerase-polymerase interaction and in activity of the RNA polymerase in vivo. More specifically, interactions via Interface I are important for efficient RNA binding but are not essential for catalytic activity and that interactions via Interface II are important for catalytic activity but not for interactions with RNA. Thus, both Interface I and Interface II are critical for RNA polymerase activity and are thus good targets for therapeutic intervention.

The present invention provides methods for identifying pharmacophores that selectively bind to the binding surfaces of the RNA polymerases involved in polymerase-polymerase interactions. These pharmacophores can be isolated using any number of techniques in the art, including but not limited to yeast two-hybrid assays and/or peptide modeling based on the structure of the RNA polymerase binding surfaces involved in oligomerization and activity.

Both homologous and heterologous interactions between RNA polymerases from virus sharing the structural aspects of Interface I and Interface II have been observed. For example, interactions between poliovirus polymerase molecules and between poliovirus polymerase and the RdRp of hepatitis A virus, a somewhat closely related RNA virus, have been observed. Thus, pharmacophores can be designed that inhibit multiple polymerase interactions, both between polymerase molecules of a single virus and between polymerase molecules from different virus. As used herein, “homologous interactions” refer to interactions between polymerases of viruses of the same virus species; “heterologous interactions” refer to interactions between polymerases of different virus species. Heterologous interactions indicate that the interfaces important for interaction of homologous polymerase subunits are also important for interaction of the subunits of other polymerses (e.g., if an Interface I is important in a homologous interaction of polymerases of Virus A, and important in heterologous interaction of polymerases of Virus A and Virus B, then Interface I is also important in homologous interaction of polymerases of Virus B).

One reason interface binding surfaces between protein molecules provide excellent targets for disruption by pharmaceuticals is these target sites are not as prone to circumvention by viral mutation.

Currently, active sites of monomeric enzymes are often targeted for inhibition by pharmaceuticals. When a mutation that confers resistance to the pharmaceutical in question arises in a population, any viral enzyme that bears that mutation is then active even in the presence of the pharmaceutical. The drug-resistant mutation is therefore genetically dominant and can exert a selective advantage even in the presence of drug-sensitive proteins that are also present in the cell. During infections with RNA viruses, this problem is especially acute for two reasons. The first is the high error rate of RNA-dependent polymerases, which misincorporate nucleotides at a rate of one mistake for every 1000-10,000 nts. polymerized (Domingo, E., C. et al, FASEB J. 10:859-864 (1996). This rate can be compared to that of DNA genomes such as those of higher eukaryotes, in which one error is made for every 10⁹ to 10¹⁰ nts polymerized. Thus, mutations of all sorts arise at a rapid rate in a population of RNA viruses, which can become fixed in the population if there is selection for them. The second, less commonly recognized feature of viral infections is that mutations usually arise in genomes that are present in cells that are co-infected with many other viral genomes.

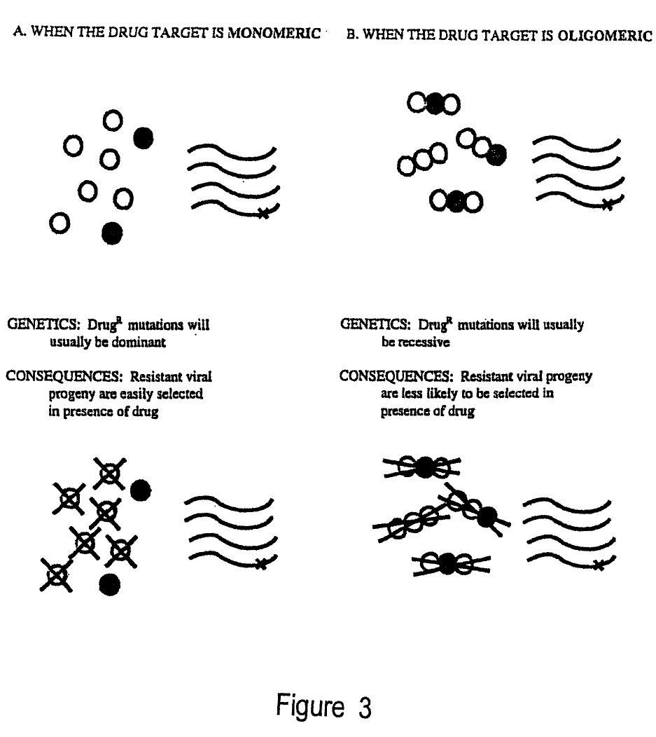

As illustrated in FIG. 3, when a drug-resistant mutation is genetically dominant, all the viral progeny of a cell that contains one mutant viral genome will enjoy a selective advantage. They will go on to infect other cells, at which time only the mutant genome and any genomes that are also present in the newly infected cells will enjoy a selective advantage.

If a protein functions in a multimeric complex, a mutation that could confer drug resistance is likely to be recessive or at best exhibit partial dominance because the drug-resistant protein must still function in a complex with wild-type, drug-sensitive proteins. The drug-sensitive members of the oligomeric complex will render the entire complex drug-sensitive. Thus, there would be less genetic selection for drug-resistant variants within infected cells. In fact, the targeting of an interaction surface between monomers of HIV protease is likely to be one of the reasons that drug-resistant HIV viruses require more mutations and arise slowly in individuals treated with the newest generation of HIV protease inhibitors (Markowitz, M., H. et al., J. Virol. 69: 701-706 (1995); Molla, A., M. et al., Nature Med. 2: 760-766 (1995)).

The pharmacophores described herein offer a therapeutic advantage over many conventional anti-viral therapeutics, since they specifically target binding surfaces of virus that are involved in the oligomerization of polymerase molecules. Thus, the pharmacophores described herein provide an improved method of inhibiting RNA polymerase activity in vivo, as the virus are less likely to circumvent the activity of the pharmacophores by mutational events.

### STRUCTURAL ASPECTS OF POLYMERASE INTERACTIONS

The present description of RNA-dependent RNA polymerases herein is made with particular reference to an exemplary RNA polymerase, the poliovirus polymerase. It is to be understood, however, that the description with reference to this molecule is exemplary, and the pharmacophores of the invention are not to be limited to the poliovirus RNA polymerase.

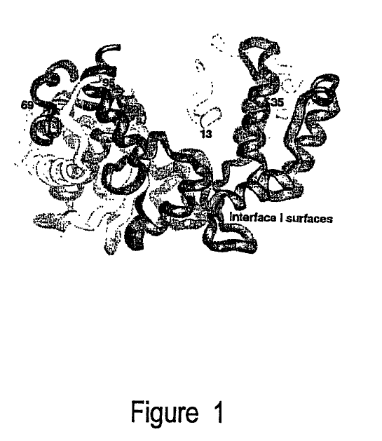

The overall structure of the poliovirus polymerase resembles that of other polymerases, which have been likened to a right hand, with the thumb at right, the fingers at left and the palm containing the most conserved sequences and the active site. The homopolymeric binding of the poliovirus is mediated by two structural elements, termed Interface I and Interface II. The structure of Interface I of the poliovirus RNA polymerase is shown in part in FIG. 1 (Hansen, J. L., et al., Structure 5: 1109-1122 (1997)). FIG. 2A illustrates the polymerase molecule interaction between poliovirus RNA polymerases within the crystal lattice via Interface I and Interface II. These interactions are much more extensive than those typically observed for crystal packing interactions, being comparable to that of antigen-antibody complexes (Janin J. and Rodier F., Proteins, 23:580-7 (1995)).

The β-turn-β structure of the poliovirus polymerase contains residues from conserved motif C, common to all polymerases (Delarue, M. V. et al., Protein Eng. 3:461-467 (1990); Poch, O., I Et al., EMBO J. 8:3867-3874 (1989). Directly adjacent to the two aspartates in this sequence is the conserved aspartate in motif A, also conserved among all polymerases (Id.). The other conserved motifs surround and support these active site residues. Like other polymerases and most resembling HIV-1 reverse transcriptase (RT), the “thumb” of the poliovirus polymerase structure is composed of several α-helices; based on homology with HIV-1 RT, the thumb is likely to serve to orient the template, or primer, or both (Jacobo-Molina, A., J. et al., PNAS USA 90:6320-6324 (1993)). The “fingers” of the poliovirus polymerase structure are very short compared to those of other known polymerases; this is undoubtedly because residues 96-182 and 266-291, likely to be found in the fingers subdomain, are disordered in the crystals.

Interestingly, the crystal structure of the poliovirus polymerase has revealed two interfaces between polymerase molecules (FIGS. 2A-2C) which may be significant in functional oligomer formation. Interface I involves residues on the side of the thumb domain (446-461) and, on a second polymerase, several peptide segments that are not contiguous in the primary sequence on the back of the palm. Interactions along Interface I would allow the formation of long fibers of polymerase, with the monomers arranged in head-to-tail orientation, as is observed in the crystal structure.

A second interface between poliovirus polymerase molecules seen in the crystal structure is Interface II, which primarily involves amino acids from the N-terminal region of the poliovirus polymerase sequence (FIG. 2). Specifically, residues 12-37, which are located close to the thumb domain, interact directly with residues 67-97, which form a helix at the base of the fingers domain (FIG. 1). The role of Interface II in the polymerase interaction has been reinforced by the following findings: mutations predicted to disrupt Interface II destroy polymerase activity but not cooperative RNA binding; low-salt crystal forms of poliovirus polymerase show half of the polymerase molecules displaying this interaction and half not, as though the formation of Interface II were a normal conformational change; and mutant forms of poliovirus polymerase in which Interface I is disrupted still interact in the yeast two-hybrid system.

The curious connectivity of N-terminal residues in the three-dimensional structure also suggest that polymerase monomers might interconnect directly across Interface II. A predicted conformation is that these sequences, 67 to 97 on the left of the image, connect to residues 12 to 37 on the right. Thus, Interface II is also a therapeutic target for pharmacophores that target intermolecular binding.

The strongest evidence that polymerase-polymerase interactions are required for polymerase activity cooperativity with respect to protein concentration displayed by polymerase activity in vitro. As polymerase concentration is increased from 1.0 to 1.5 μM, the amount of extended substrate increased from approximately 10% to 90%. Mutations predicted to disrupt Interface I, e.g., AL28, are lethal to the virus (See Diamond, S. E. and Kirkegaard K. J. Virol. 68:863-876 (1993), as are mutations predicted to disrupt Interface II, e.g., AL-2 (K38,E39), AL-25 (K405, D406) (Diamond, S. E. and Kirkegaard K. J. Virol. 68:863-876(1993)) and AL33 (V33, F34).

The RNA-dependent RNA polymerase sequences from other positive-strand viruses also display long N-terminal segments, with the flaviviruses displaying even longer segments N-terminal of Motif A than poliovirus. The functions of these N-terminal sequences are likely to be unique to RNA-dependent RNA polymerases. Although the long N-terminal sequences only display recognizable homology among the most closely related positive-strand RNA viruses, the function and even details of their structure are believed to be widely conserved. The N-terminal sequences of other positive-strand RNA viruses, like those of poliovirus, are believed to be involved in the formation of polymerase-polymerase interactions.

The conserved structures of Interface I and Interface II of various virus thus offer novel targets for antiviral drug discovery.

### DESIGN AND TESTING OF PHARMACOPHORE OF THE INVENTION

The pharmacophores of the present invention can be identified using the 3D structural aspects of the binding surfaces of RNA polymerases. Two exemplary methods for identifying structures that selectively bind to one or more viral RNA polymerases are described as follows.

Two-Hybrid System of Saccharomyces cerevisiae and Interactions between RNA Polymerases

The two-hybrid system in S. cerevisiae detects protein-protein interactions in yeast cells by monitoring the activation of transcription of a reporter gene following the reconstitution of an active transcription factor via those protein-protein interactions. This method can be used to identify both homologous and heterologous interactions between polymerases of several positive-strand RNA viruses representative of picornaviruses, alphaviruses, flaviviruses, etc. and to screen both structure-based peptides and combinatorial libraries of peptides for those that inhibit all observed interactions. The two-hybrid system can identify pharmacophores, such as peptides or combinations of peptides, that will target a wide spectrum of RNA viral infections.

For example Yang et al. (Nucl. Acids Res. 23:1152 (1995)) describes a method for identifying peptides that bind to proteins by screening a library of DNA plasmids in which 16 random amino acids were fused to the extreme C-terminus of the transcriptional activation domain of a “prey”-encoding plasmid. This library of prey plasmids was then screened for those that interacted with a bait plasmid that contained the protein sequences fused to a DNA-binding domain. The 16 random amino acids were encoded by the sequence (NNK)₁₆, in which “N” is an equimolar mixture of G, C, T and A and “K” is an equimolar mixture of G and T. The inclusion of only two of the four possible nucleotides at the “wobble” position allowed every amino acid to be encoded but reduced the complexity of the library and minimized the introduction of UAA and UGA stop codons. This method can be used to identify sequences that bind to Interface I, Interface II, or other binding surfaces of a viral RNA-dependent RNA polymerase.

In a different strategy, Colas et al. (Nature 380:548-550 (1996)) prepared a library in which a sequence of 60 randomized nts., encoding 20 random amino acids, was cloned into a region of bacterial thioredopsin known to be tolerant to insertions of foreign sequence. Their rationale was that such “conformationally constrained” random peptide sequences might reduce the entropic cost of peptide binding and thus increase the affinity of the isolated peptides for their target. The library was then screened to test the ability of any of these insertion-containing thioredoxin proteins to interact with another protein in the two-hybrid system. This methodology can be used to identify insertion-containing thioredoxin proteins to interact with Interface I, Interface II, and/or other binding surfaces of an RNA-dependent RNA polymerase that are involved in polymerase-polymerase interaction and activity.

Specific examples of two-hybrid methodologies that can be used to identify pharmacophores are McBride, A. E et al., Proc. Natl. Acad. Sci. USA 93, 2296-2301 (1996) and Hope, D. A et al.., J. Virol. 71:9490-8 (1997). Other two hybrid methodologies can also be used, as will be apparent to one skilled in the art upon reading the present disclosure.

The use of a two-hybrid system to identify pharmacophore of the invention is particularly advantageous, since the binding surfaces, e.g. Interface I or Interface II, can be assayed separately. Thus, by using Interface I as the structure of the “bait”, the “prey” pharmacophores can be identified that are specific to Interface I. Similarly, by using the structure of Interface II as the “bait”, pharmacophores specific to Interface II can be identified.

Rational Design of Pharmacophores

The shape that the viral RNA-dependent RNA polymerase adopts at its binding surfaces when bound to another polymerase, the biological shape, is an essential component of its biological activity. This shape, and any specific interactions such as hydrogen bonds, can be exploited to derive predictive models used in rational drug design. These can be used to optimize lead compounds, design de novo compounds, and search databases of existing compounds for novel structures possessing the desired biological activity. In order to aid in the discovery of useful pharmacophores for the interface binding surface, these models must make useful predictions, relate chemical structures to activity, and be confidently extrapolated to chemical classes beyond those used for model derivation.

Pharmacophore models (e.g., BioCAD incorporated herein by reference) model activity in terms of the positions of a small number of atoms of particular functional groups. This overcomes many of the problems of traditional QSAR models. U.S. Pat. No. 5,025,388 to Kramer et al. provides for comparative molecular field analysis (COMFA incorporated herein by reference) methodology. In accordance with this methodology the 3-dimensional structure for each molecule is placed within a 3-dimensional lattice and a probe atom is chosen, placed successively at each lattice intersection and the stearic and electrostatic interaction energies between the probe atom and the molecule are calculated for all lattice intersections. The energies are listed in a 3-dimensional-QSAR table. A field fit procedure is applied by choosing the molecule with the greatest biological activity as the reference in conforming the remaining molecules to it.

U.S. Pat. No. 5,436,850 (incorporated herein by reference in its entirety) describes a computer-assisted method for identifying protein sequences that interact with known protein structures. The method uses a known three-dimensional protein structure and determines three key features of each residue's environment within the structure: (1) the total area of the residue's side-chain that is buried by other protein atoms, inaccessible to solvent; (2) the fraction of the side-chain area that is covered by polar atoms (O, N) or water, and (3) the local secondary structure. Based on these parameters, each residue position is categorized into an environment class. In this manner, a three-dimensional protein structure is converted into a one-dimensional environment string, which represents the environment class of each residue in the folded protein structure. A 3D structure profile table is then created containing score values that represent the frequency of finding any of the 20 common amino acids structures at each position of the environment string. These frequencies are determined from a database of known protein structures and aligned sequences. The method determines the most favorable alignment of a target protein sequence to the residue positions defined by the environment string, and determines a “best fit” alignment score for the target sequence.

The methodology disclosed within U.S. Pat. No. 5,526,281 (incorporated herein by reference in its entirety) is particularly useful for the generation of pharmacophores using the information provided herein. In particular, the 3-dimensional coordinates shown within FIGS. 2A-C can be applied to the polymerase molecules of various virus. This methodology is useful for the generation of pharmacophores of the present invention because, in many binding interactions between molecules, not all the characteristics of the molecule considered are of equal importance. As shown within FIGS. 2A-C, the regions of the viral polymerase not within the interface binding surfaces are not as advantageous as targets for polymerase activity, as these mutations will not affect polymerase-polymerase interaction and thus the virus are more likely to compensate for these mutations. This program can focus on the regions of the polymerase involved in the polymerase-polymerase interaction while minimizing the areas that of are of little importance with respect to the surface of the interface binding surfaces. The approach disclosed within the '281 patent thus allows the user to focus on the salient features of the molecule.

The first step in the '281 method for generating pharmacophores involves the selection of a pose. A pose of a molecule is defined by its confirmation (internal torsional angles of the rotatable bonds) and orientation (the rigid rotations and translations). The negative image of the pose can also be generated and as such represents a pose for the corresponding binding surface of homologous and/or heterologous interactions with other viral polymerases. The negative image and other possible pharmacophores can be generated using software available such as Catalyst™ from BioCad, Foster City, Calif. and, Batchmin™ available from Columbia University, New York City, N.Y. (both of which are incorporated herein by reference). These programs take into consideration various properties including physical and chemical properties, Shape, electrostatic interaction, solvation and biophysical properties.

Other methods for generating pharmacophores of the present invention are disclosed within U.S. Pat. Nos. 5,884,230, 5,307,287, and 5,434,796 each of which are incorporated herein by reference in their entirety.

In general, then, the invention contemplates computer comprising a representation of a pharmacophore of the invention in computer memory. In this embodiment, the pharmacophore is represented as a three-dimensional array of points defining a specific shape and volume. The three-dimensional array of points is generally an aggregate average shape of a molecule (or a plurality of molecules) when that molecule optimally interacts with Interface I or Interface II of an RNA-dependent RNA polymerase in a manner that results in disruption or prevention of polymerase-polymerase interactions. This three-dimensional array of points can be represented by a coordinate system configured in computer memory. The computer or computer system can thus be used to design a molecular structure that can disrupt or prevent polymerase-polymerase interactions as described herein, and can further be used to screen candidate molecular structures for the ability to disrupt or prevent such polymerase-polymerase interactions.

### INTRODUCTION OF PHARMACOPHORES INTO INFECTED CELLS

The pharmacophores of the invention may be delivered in various forms that are able to enter into an infected cell and to affect the activity of an RNA-dependent RNA polymerase.

For example, in one embodiment of the invention, pharmacophores can be introduced as exogenous genetic entities using any of a variety of different approaches known to those skilled in the art. Such approaches include both ex vivo and in vivo therapy. For a review of these approaches, see Ledley, Pharm Res, 13:1595-614 (1996). The pharmaceutically acceptable carrier for such genetic entities are generally nucleic acid vectors. Numerous vectors are presently used by those in the field, and could be used to introduce the compounds of the invention. These include, but are not limited to, viral-based vectors, particle-mediated gene delivery, calcium phosphate delivery, liposome-mediated gene delivery, ligand/DNA conjugates, episomes, extrachromosomal replicating vectors. For a review of such vectors, see Cooper, Semin Oncol 23:172-187 (1996); see also Calos, Trends Genet 12: 463-6 (1996).

In one embodiment, the pharmacophore of the invention is a peptide that is delivered to infected cells and/or organisms by modification of the pharmacophore to include a motif that allows entry into cells and/or delivery to the nucleus once the pharmacophore has entered into a cell. For example, pharmacophore may be designed to be fuse or conjugated to a small peptide derived from the tat protein of HIV that enters cells directly, carrying with it any associated molecules. In another example, the pharmacophore of the invention may contain a structural motif that allows the pharmacophore to be directed to the site of cellular activity of the RNA-directed RNA polymerase. Other modifications to the pharmacophore can also be made, as will be apparent to one skilled in the art upon reading the present disclosure.

Further, pharmacophores can be made as conjugate molecules wherein the pharmacophore is linked in some manner to a label, e.g., fluorescent, radioactive and enzyme labels. By forming such conjugates, the compound of the invention act as biochemical delivery systems for the label so that a site of infection can be detected.

### EXEMPLARY VIRAL RNA-DEPENDENT RNA POLYMERASE TARGETS

One aspect of the invention is to provide pharmacophore peptide compounds that can inhibit replication by many different positive-strand viruses, even those whose identity is presently unknown. To identify such peptides, polymerase coding regions from a variety of positive-strand viruses can be cloned into plasmids for expression in E. coli, for testing in the described two-hybrid methods, and/or for modeling of the polymerase binding regions. This technique has been successful with several representative positive-strand RNA viruses as described below, and some information about each of these is provided. Nucleic acid and amino acid sequences of the RNA-dependent RNA polymerase of many RNA viruses are known in the art.

Sequence alignments of picornaviral polymerases reveal a significant degree of conservation among amino acid residues within and around both Interfaces I and II, suggesting that these regions of interaction are generally important in picornaviruses. Picronaviruses define a family of small RNA viruses, which includes the subgroup enteroviruses, which in turns includes polioviruses, coxsackie viruses (Group A and Group B), echoviruses (which can be fatal to newborns), rhinoviruses (which cause the common cold), hepatitis A, and enteroviruses. Coxsackieviruses, which can cause lethal myocarditis in humans, have become of particular interest recently due to the outbreaks of foot-and-mouth (or hoof-and-mouth) disease in livestock (e.g., sheep and cattles), which is commonly caused by coxsackievirus A16.

The worldwide effort to eradicate poliovirus by the year 2000 or very soon thereafter will soon lead to the cessation of worldwide vaccination. For most picornaviruses, infectious cDNAs exist which can readily be used to generate wild-type or mutant infectious virus. It has been shown recently that coding regions can be inserted into, and expressed from, the poliovirus genome (Andino, R. et al., Science 265:1448-1451(1994)); this is likely to be the case for other picornaviruses as well. A good mouse model requires the use of transgenic mice that express the poliovirus receptor (Koike, S. et al. PNAS USA 88:951-955 (1991); Ren, R. B. et al., Cell 63:353-362 (1990)). Poliovirus type 1 Mahoney is a model virus, since the structure of the RNA-dependent RNA polymerase is known and the in vitro polymerase activity of the PV1-3Dpol coding region has been studied extensively.

Enterovirus 71 is a newly emergent enterovirus whose symptoms are indistinguishable from those formerly observed upon poliovirus infection. Immunity to poliovirus does not confer immunity to enterovirus 71. The genome of enterovirus 71 was recently sequenced, and it shows no more sequence similarity to poliovirus than to any other enteroviruses. The polypeptide predicted to be the enterovirus 71 RNA-dependent dependent RNA polymerase is E71-3D, which can be tested for polymerase activity and polymerase-polymerase interactions in vitro and in vivo.

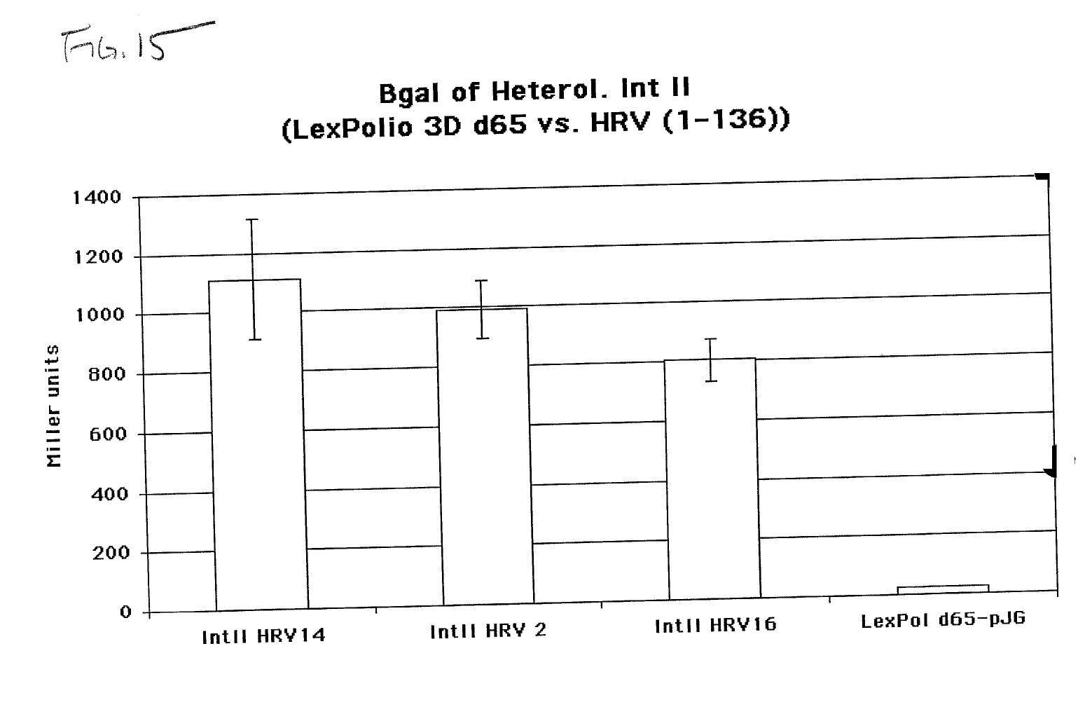

Rhinovirus is an example of a picornavirus closely related to poliovirus that can be delivered by a respiratory or contact route. The polypeptide predicted to be the HRV14 RNA-dependent RNA polymerase, HRV-3D, displays for polymerase activity in vitro and polymerase-polymerase interactions in the yeast two-hybrid system. These interactions are homologous with poliovirus polymerase interactions (FIG. 14) at both Interfaces I (data not shown) and II (FIG. 15).

Alphaviruses, which are members of the Togaviridae family of RNA positive strand viruses, are another group of viruses for which pharmacophores of the invention are contemplated. Sindbis virus is the most-studied member of the alphaviruses, a group which includes many serious human pathogens such as Eastern equine encephalitis virus and Venezuelen equine encephalitis virus. Sindbis virus causes no serious pathology in adult humans, although it must circulate in human populations, because humans with antibodies against Sindbis virus have been found throughout the world. Presumably, these Sindbis virus infections occur via mosquito transmission from avian populations in which Sindbis virus is enzootic. Sindbis virus grows well in tissue culture, and a mouse model is available, in which infant rodents given virus intraperitoneally or intracerebrally develop lethal encephalitis (Sherman, L. A. and D. E. Griffin. J. Virol. 64:2041-2046 (1990)). The polypeptide predicted to be the RNA-dependent RNA polymerase, Sin-nsP4 shows RNA-dependent RNA polymerase activity in mammalian cells when expressed independently of other viral proteins (Lemm, J. A. and C. M. Rice J. Virol. 67: 1905-1915 (1993)), and can be tested for polymerase activity and polymerase-polymerase interactions in vitro and in vivo. Sindbis virus polymerase interacts with both poliovirus and rhinovirus polymerases in the two-hybrid system, arguing that at least one of intermolecular interfaces is conserved from picornaviruses to apthoviruses (FIG. 14).

Yellow fever virus is the prototype member of the flavivirus family of positive-strand RNA viruses, another family of viruses contemplated by the invention. The virus is transmitted via mosquitoes between human or non-human primates; mortality rates can be as high as 20% in a human population during an urban epidemic. Infectious transcripts can be made from full-length cDNAs of an attenuated vaccine strain, 17D (Rice 1989). Replication in tissue culture is slow but assayable. In mouse models for yellow fever virus infection, virus replication can be monitored by quantifying viral titer in the spinal fluid and in mouse tissues (Schlesinger, J. J. et al. J. Gen. Virol. 77: 1277-1285 (1996). The polypeptide predicted to have RNA-dependent RNA polymerase activity, YFV-ns5, can be tested for polymerase activity and polymerase-polymerase interactions in vitro and in vivo.

Dengue fever virus is also a member of the flavivirus family. The predominant disease caused by Dengue is a self-limiting fever, but it can, in up to 20% of the infected population, lead to lethal hemorrhagic fever. There is some antigenic cross-reactivity between Dengue and yellow fever virus. Like yellow fever virus, Dengue virus is spread via arthropod vectors, usually mosquitoes, to humans and other primates. The predicted polymerase coding region, Dengue-ns5pol, has been expressed in E. coli and shown to display RNA-dependent RNA polymerase activity (Tan, B.-H. et al., Virol. 216:317-325 (1996)). Dengue-ns5pol can be tested for polymerase activity and polymerase-polymerase interactions in vitro and in vivo.

The structure of the RNA polymerase of hepatitus C virus (HCV), a positive-strand RNA virus, has been reported by three different labs (Ago et al., Structure Fold Des. 7:1417-26 (1999); Bressanelli S. et al. PNAS USA 96:13034-9 (1999); Lesburg C. A. et al., Nat Struct Biol 6:937-43 (1999)). The structure of the HCV polymerase is overall similar to that of poliovirus polymerase, especially in the palm subdomain, but also exhibits several interesting differences. In the HCV polymerase, the N-terminal polypeptide segment does not interact in the active site cleft as in poliovirus polymerase, rather a peptide loop from the HCV polymerase thumb subdomain (residues 441-456), which is not present in poliovirus polymerase, extends down into its active site cleft, apparently replacing the N-terminal strand interaction of poliovirus polymerase. Recent evidence shows that, counter to reports that no extensive protein-protein contacts are observed in HCV crystals, HCV may in fact involve higher order polymerase complexes such as those observed for poliovirus. Thus, HCV polymerse can be tested for polymerase activity and polymerase-polymerase interactions in vitro and in vivo.

Candidate inhibitory pharmacophores based on the structure of various RNA-dependent RNA polymerases are tested for their antiviral activities in systems such as cell culture. Due to the simple infectious cycle of positive-strand RNA viruses, infections can be initiated using either cloned DNA or RNA transcripts templated from cloned DNA for most of the positive-strand RNA viruses for which such experiments have been attempted. Furthermore, cDNA copies of the genomes of poliovirus, rhinovirus and especially Sindbis virus have proven to be useful vectors for the delivery of unrelated proteins to infected cells. Antiviral compounds that targeted a broad spectrum of positive-strand RNA viruses can be of great therapeutic importance.

### THERAPEUTIC USE OF ANTI-VIRAL PHARMACOPHORES

When used in the therapeutic treatment of viral disease, an appropriate dosage of one or more pharmacophores of the invention may be determined by any of several well established methodologies. For instance, animal studies are commonly used to determine the maximal tolerable dose, or MTD, of bio-active agent per kilogram weight. In general, at least one of the animal species tested is mammalian. Those skilled in the art regularly extrapolate doses for efficacy and avoiding toxicity to other species, including human. Additionally, therapeutic dosages may also be altered depending upon factors such as the severity of infection, and the size or species of the host.

Where the therapeutic use of the presently described pharmacophores is contemplated, a composition containing such pharmacophores is preferably administered via a pharmaceutically acceptable carrier, via oral, intranasal, rectal, topical, intraperitoneal, intravenous, intramuscular, subcutaneous, subdermal, transdermal, intertracheal methods, or the like.

Typically, but not necessarily, the preferred formulation for a given antiviral composition is dependant on the location in a host where a given virus would be expected to initially invade, or where a given virus would be expected to colonize or concentrate. For example, where the virus causes a systemic infection, it would be preferable to administer the anti-viral composition by oral dosing or parenteral means. In another example, where the virus to be treated infects the sinuses, an intranasal administration would be preferable. In yet another example, pulmonary infections may be treated both parenterally and by direct application of suitably formulated compositions to the lung by inhalation therapy.

One of ordinary skill will appreciate that, from a medical practitioner's or patient's perspective, virtually any alleviation or prevention of an undesirable symptom (e.g., symptoms related to the presence of virus in the body) would be desirable. Thus, the terms “treatment”, “therapeutic use”, or “medical use” used herein shall refer to any and all uses of the claimed pharmacophore which remedy a viral disease state or symptoms, or otherwise prevent, hinder, retard, or reverse the progression of disease or other undesirable symptoms in any way whatsoever.

Preferably, animal hosts that may be treated using the pharmacophores of the present invention include, but are not limited to, invertebrates, vertebrates, birds, mammals such as pigs, goats, sheep, cows, dogs, cats, and particularly humans.

The presently described pharmacophores may be formulated with a variety of physiological carrier molecules. For example, the presently described pharmacophores may be complexed with molecules that enhance their ability to enter the target cells. Examples of such molecules include, but are not limited to, carbohydrates, polyamines, amino acids, peptides, lipids, and molecules vital to bacterial growth. For example, the pharmacophores may be combined with a lipid, cationic lipid, or anionic lipid. The resulting pharmacophore/lipid emulsion, or liposomal suspension may effectively increase the in vivo half-life of the pharmacophore. The use of cationic, anionic, and/or neutral lipid compositions or liposomes is generally described in International Publications Nos. WO90/14074, WO 91/16024, WO 91/17424, and U.S. Pat. No. 4,897,355, herein incorporated by reference.

The composition of the liposome is usually a combination of phospholipids, particularly high-phase-transition-temperature phospholipids, usually in combination with sterols, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidyl-glycerol, phosphatidylcholine, phosphatidylserine, phosphatidylethanolamine, sphingolipids, cerebrosides, and gangliosides. Particularly useful are diacylphosphatidyl-glycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticulo-endothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization. In this manner, specific cell types that are infected can be targeted for introduction of the anti-viral pharmacophore.

Pharmaceutical compositions containing the pharmacophores of the invention in admixture with a pharmaceutical carrier can be prepared according to conventional pharmaceutical compounding techniques. The carrier may take a wide variety of forms depending on the form of the preparation desired for administration, e.g., intravenous, oral, topical, aerosol (for topical or inhalation therapy), suppository, parenteral, or spinal injection.

In preparing the compositions in oral dosage form, any of the usual pharmaceutical media may be employed, such as, for example, water, glycols, oils, alcohols, flavoring agents, preservatives, coloring agents and the like in the case of oral liquid preparations (such as, for example, suspensions, elixirs, and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated and enteric-coated by standard techniques.

For parenteral application by injection, preparations may comprise an aqueous solution of a water soluble, or solubilized, and pharmaceutically acceptable form of the pharmacophore in an appropriate saline solution. Injectable suspensions may also be prepared using appropriate liquid carriers, suspending agents, agents for adjusting the isotonicity, preserving agents, and the like. Actual methods for preparing parenterally administrable compositions and adjustments necessary for administration to subjects will be known or apparent to those skilled in the art and are described in more detail in, for example, Remington's Pharmaceutical Science, 15th Ed., Mack Publishing Company, Easton, Pa. (1980), which is incorporated herein by reference. The presently described pharmacophores should be parenterally administered at concentrations below the maximal tolerable dose (MTD) established for the pharmacophores.

For topical administration, the carrier may take a wide variety of forms depending on the preparation, which may be a cream, dressing, gel, lotion, ointment, or liquid.

Aerosols are prepared by dissolving or suspending the pharmacophore in a propellant such as ethyl alcohol or in propellant and solvent phases. The pharmaceutical compositions for topical or aerosol form will generally contain from about 0.01% by weight (of the pharmacophore) to about 40% by weight, preferably about 0.02% to about 10% by weight, and more preferably about 0.05% to about 5% by weight depending on the particular form employed.

Suppositories are prepared by mixing the pharmacophore with a lipid vehicle such as theobroma oil, cacao butter, glycerin, gelatin, or polyoxyethylene glycols.

### EXAMPLES

The following examples are put forth so as to provide those of ordinary skill in the art with a complete disclosure and description of how to practice the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Interface I Mutational Analysis

Polymerase-polymerase interactions at Interface I involve two distinct and separate surfaces of the polymerase molecules. One surface is positioned at the bottom of the thumb subdomain and the other at the back of the palm subdomain (See FIGS. 1 and 2). Importantly, these interactions do not give rise to a discrete dimeric, trimeric, etc. structural units, rather the Interface I interactions give rise to a directional fiber in which the polymerase molecules associate in a head to tail fashion and which might vary greatly in length.

The Interface I interactions involve 21 direct amino acid side chain interactions, at least 5 water mediated interactions and one direct backbone-backbone hydrogen bond. The interactions at Interface I can be divided into two distinct regions. The first region (at the top of Interface I in FIG. 2C) centers around L446 which extends from the surface of one polymerase molecule and into a hydrophobic pocket in the adjacent polymerase molecule. The hydrophobic pocket of this second molecule is formed from two separate peptide loops which immediately precede and follow the conserved poliovirus polymerase C motif (Hansen, J. L. et al.,. Structure 5:1109-1122 (1997)). Numerous direct as well as water mediated interactions also occur between the residues in these two loops and the residues flanking L446. The second region of interaction at Interface I involves two α-helices (the C-terminal α-helix of one molecule and the motif D α-helix of the second molecule) that pack together at an angle of approximately 90°. Almost all of the surface exposed amino acid side chains on both of these helices interact across this interface. These interactions include R455 from one helix which hydrogen bonds with D349 from the helix of the adjacent polymerase molecule as well as L342 from one molecule, which packs against a hydrophobic patch on the other polymerase molecule.

To evaluate potential functional roles for interactions at Interface I, several mutations were constructed at Interface I that should attenuate and disrupt the protein-protein interactions at this interface. Seven different mutant polymerases containing amino acid substitutions along both surfaces of Interface I were generated: these are L342A, D349R, L446A, R455D, R456D, an L446A: R455D double mutant and an R455A: R456A double mutant that had been previously reported (Diamond, S. E. and Kirkegaard K. J. Virol. 68:863-876(1993)). )). The nomenclature used here is as follows: the first letter refers to the amino acid residue present in the wild-type polymerase, the number refers to the position of that residue, and the second letter refers to the amino acid residue in the mutant polymerase.. These mutations were widely dispersed along both surfaces of Interface I in order to reduce difficulties in interpretation arising from potentially local specific effects.

All mutant forms were introduced into the Mahoney type 1 strain by PCR mutagenesis and the mutations were verified by DNA sequencing. WT and mutant polymerases were expressed using a T7 based expression system in E.coli BL21(DE3) pLysS. Cells containing the expression plasmid were grown in 2×YT media at 37° C. to an O.D.₆₀₀ of −0.2. The cells were then cooled to room temperature (RT), grown to an O.D.₆₀₀ of −0.4, induced with 0.5 mM IPTG, and then grown 16 to 18 hours at RT. The polymerase was purified as described previously (Hansen, J. L. et al., Structure 5:1109-1122 (1997)) except that an FPLC S-Sepharose column was used instead of using a gravity S-Sepharose column. The protein was eluted from the S-Sepharose column using a linear gradient of NaCl from 0.1 M to 0.38 M NaCI. Typical yields of the WT and mutant polymerases are 12 mg per 10 g of cells (wet weight). In mass spectroscopic analysis, only the N-terminal endoproteinase Lys-C fragment missing the N-terminal Met was observed, indicating that the N-terminal Met was effectively removed. Protein purified in this manner was indistinguishable from protein purified as a 3CD cleaved product.

All seven of these mutant polymerases are stable, soluble proteins that expressed at levels comparable to that of the wildtype protein in E. coli. Interestingly, each of the Interface I mutant polymerases exhibits a significantly greater solubility than the WT polymerase. For example, the L446A: R455D double mutant is soluble at concentrations of up to 20 mg/mL in solutions containing 50 mM NaCl, pH 7.5, while the WT protein is soluble only to <0.2 mg/mL in the same conditions.

### Example 2

RNA Binding of Interface I Mutants

The Interface I mutations were also shown to affect the RNA binding properties of poliovirus polymerase. Each of the Interface I mutant polymerases binds the 0.20 RNA and the 5.20 RNA less tightly than the WT polymerase.

In polymerase structures with bound primed template nucleic acid (Doublie S. and Ellenberger T. Curr Opin Struct Biol. 8:704-12 (1998); Steitz T. A. J Biol Chem. 274:17395-8 (1999)), the single strand template region wraps across the front of the fingers subdomain which is a region that is disordered in the structure of poliovirus polymerase. Thus, this portion of the structure may fold together with the single strand template region and give rise to different binding properties for RNA substrates containing a single strand template region. The 0.20 RNA substrate, therefore, would emphasize interactions with the double strand region of primed template RNA substrates. The N-terminal mutants, which are not involved in interactions at Interface I, bind RNA as well as the WT polymerase, indicating that the binding effects observed for the Interface I mutants are specific to Interface I.

RNA binding by the WT and mutant proteins was evaluated by nitrocellulose filter binding (Wong I. and Lohman T. M. PNAS USA. 90:5428-32 (1993)) using two different RNA substrates, a 20 nucleotide self-complimentary RNA (0.20 RNA) that forms a 20 base pair (bp) duplex, and a 25 nucleotide self-complementary RNA (5.20 RNA) that anneals to form a 20 by duplex region and five nucleotide 5′ single strand extensions.

The 0.20 RNA and 5.20 RNA were generated by T7 transcription. The primer and template DNAs were purchased from Operon Technologies. Inc. The T7 transcription reaction contained: 0.2 μM 5.20 template DNA (5′GGCAGCTGCATGCAGCTGCCGCGCCTATAGTGAGTCGTATTAATTTCG-3′; SEQ ID NO:1) or 0.2 μM 0.20 template DNA (5′-GGCAGCTGCATGCAGCTGCCTATAGTGAGTCGTATTAATTTCG-3′; SEQ ID NO:2), 0.22 μM T7 DNA promoter (5′CGAAATTAATACGACTCACTATAT-3′ SEQ ID NO:3), 40 mM Tris pH 8.0, 25 mM MgOAc, 0.01% Triton X-100, 10 mM DTT, 1 mM spermidine, 2 mM each of ATP, GTP and UTP, 1 mM CTP and 0.12 u ci\μL [α-³²P]-CTP (3,000 Ci/mmol, NEN). After incubation at 37° C. for 3 hr, NaOAC pH 5.2 was added to 100 mM and the sample was put on ice for 20 min. Precipitate was removed by centrifugation. EtOH (2½ volumes) was then added and the RNA was collected by centrifugation, washed twice with 70% EtOH, dried, and resuspended with 50 μL 1/10 T.E. (1 mM Tris-HCL pH 8.0, 0.1 mM EDTA) and 50 μL 90% formamide, 30 mM EDTA and bromophenol blue. This sample was loaded onto a 16% polyacrylamide (19:1) gel containing 8.3M urea.

Following electrophoresis, the bands were visualized with UV shadowing and the desired bands were cut out and eluted for 2 days in approximately 2 mL of 20 mM NaOAc pH 5.2, 0.02% SDS and 0.2 mM EDTA at 4° C. The polyacrylamide was then removed using a spin filter screening column (Fischer Scientific), the samples were concentrated to 400 μL, and the RNA was precipitated with ethanol. The RNA pellet was dried and resuspended with 50 μL of 1 mM Tris-HCL pH 8.0, 0.1 mM EDTA. Concentrations were determined by A₂₆₀ using the average RNA extinction coefficient of 0.11 mM/nucleotide.

RNA binding was evaluated using a nitrocellulose filter binding assay. The binding reactions contained 25 mM Mes pH 6.5, 2.5 mM Mg(OAc)₂, 2 mM DTT, 10% glycerol, 40 pM ZnSO₄ and 1 mM of each GTP, CTP and UTP, 10 nM [α-³²P] labeled RNA and polymerase at various concentrations from 7.5 nM to 3.0 μM. The 30 μL binding reactions were incubated for 15 min on ice and then loaded onto a 96 well filter apparatus containing layers of (i) nitrocellulose membrane to bind all RNA bound by protein,(ii) a 25 μM filter paper (Whatman) for separation of layers, (iii) a positively charged nylon membrane (HybondN+,Amersham) to capture all unbound RNA, and (iv) Gel Blot paper (GB002, Schleicher & Schuell). All filters and paper were equilibrated in 50 mM Mes pH 6.5 and 10 mM EDTA for at least 30 min. prior to use. The amount of RNA bound to each membrane was quantitated using a phosphorimager.

The solution conditions used to evaluate RNA binding and primed template RNA utilization were different from standard assay conditions for poliovirus polymerase. Standard conditions generally utilize very low salt concentrations (<5 mM NaCl) as well as an excess of primed template RNA so that substrate is never limiting (Paul et al., (1994) J. Biol Chem. 269:29173-19181. Assays to evaluate RNA binding by the polymerase, however, require limiting RNA substrate and higher salt concentrations to prevent precipitation at high polymerase concentrations.

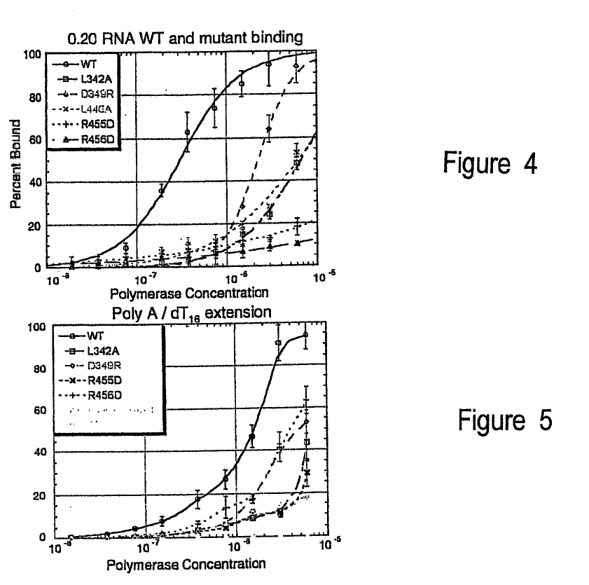

Binding of the WT and the Interface I mutant polymerases to duplex 0.20 RNA as a function of polymerase concentration is shown in FIG. 4. Each of the Interface I mutant polymerases binds 0.20 RNA much more weakly than the WT polymerase. The Kds are 8 to >20 fold higher for each of the singly mutant polymerases. The D349R mutation was least disruptive, the L342A and L446A mutations were more disruptive, and the R455D and R456D mutations were most disruptive.

Likewise, each of the Interface I mutant polymerases binds the 5.20 RNA more weakly than the WT polymerase. Although these differences are not as dramatic as for the 0.20 RNA, perhaps because of interactions with the single strand region of this template, the Kds of the mutant polymerases are consistently and reproducibly higher than that of the WT polymerase. For binding the 5.20 RNA, the L342A mutant was least disruptive, the D349R mutation was slightly more disruptive and the R456D mutation was most disruptive. The doubly mutant AL28 and L446A:R455D polymeses bound the 5.20 RNA with lower affinity than the singly mutant polymerases. In contrast, poliovirus polymerase deletion mutant (D6) in which the first 6 N-terminal residues are removed, disrupting Interface II and destroying polymerase activity, binds the 5.20 RNA indistinguishably from the WT polymerase.

### Example 3

RNA Substrate Utilization of Interface I Mutants

RNA extension analyses show that the Interface I mutant polymerases incorporate NTPs into primed template RNA substrates less efficiently than the WT polymerase (FIG. 5). This result was observed for two different RNAs, poly-A primed with oligo dT₁₆ as well as the 5.20 RNA, indicating that this effect is not specific to the substrate used. Poly-A and dT₁₆ were purchased from Pharmacia Biotech.

To evaluate for the ability of the mutant polymerases to efficiently extend primed-template RNA substrates, two different substrates were utilized, the 5.20 RNA which was used in the binding studies and poly-A primed with oligo dT₁₆ to insure that observed effects were not unique to the specific template utilized.

For the poly A-oligo dT₁₆ substrate, the reactions were carried out in 25 mM Mes pH 6.5, 2.5 mM Mg (OAc)₂, 50 μM UTP and 50 mM NaCl with excess polymerase. These conditions differ from standard polymerase activity assay conditions as will be discussed later. The results are shown in FIG. 5. It is apparent from this analysis that polymerases containing mutations at Interface I incorporate nucleotides to a lesser extent than WT polymerase, especially at low polymerase concentrations. Importantly, however, each of the singly mutant polymerases is able to achieve a significant level of incorporation at high concentrations of polymerase, with D349R, R456D and L342A showing the highest levels of incorporation. Incorporation of radiolabel by the doubly mutant polymerases is further reduced from that of the singly mutant polymerases.

Elongation of the 5.20 RNA was evaluated using native polyacrylamide gels so that the actual fraction of substrate utilized in the extension reactions could be observed directly. Extension was evaluated for WT and mutant polymerase at concentrations of 75 nM, 375 nM and 1500 nM. Native polyacrylamide gels resolve three RNA species corresponding to unelongated duplex 5.20 RNA, a duplex RNA with one of the two 3′ sites elongated, and a duplex RNA with both 3′ sites of elongated. The identity of these bands was verified by isolating the bands from native gels, denaturing the products by boiling and evaluating them using a denaturing polyacrylamide gel (data not shown). This analysis shows that although the Interface I mutant polymerases utilize the 5.20 RNA substrate less efficiently than wild type polymerase under these conditions, at high concentrations the mutant polymerases are able to at least partially overcome this effect and show substantial enzymatic activity.

### Example 4

In Vivo Analysis of Interface I Mutations

Each of the Interface I mutations (L342A, D349R, L446A, R455D, R456D and L446A: R455D) was also introduced into the 3D coding region of the poliovirus genome. These results indicate that mutations that disrupt polymerase-polymerase interactions in vitro also affect the viability of poliovirus in vivo. The distributed nature of the mutations along both sides of Interface I argue against specific local effects. Thus it is very likely that pharmacophores to these sites of the polymerase can be useful therapeutics in vivo.

Insertion of Interface I mutations into the poliovirus genome was accomplished using PCR mutagenesis. The PCR fragment and the T7pGEMpolio plasmid were digested with 10 Units Bgl II and EcoRl restriction enzymes for 1 hour at 37° C. Ligation into the unique Bgl II and EcoRl sites in the plasmid was done with 5 Units T4 DNA Ligase at 18° C. for 12 hours.

Two different cloned cDNAs corresponding to each Interface I mutation or set of Interface I mutations were subcloned into the poliovirus cDNA-containing plasmid T7Gem polio (Sarnow P., J Virol. 63:467-70 (1989)). KJT7 cells (Nugent et al., (1999) J. Virol. 73:427-435) were transfected with mutant or wild-type plasmid DNA using LipofectaminePLUS (GIBCO) following the manufacturer's instructions. Transfected cell monolayers were overlaid with Dulbecco's modified Eagle medium containing 10% calf serum and 1% agar. Plaques were allowed to develop for 72 h at 32.5° C. or 48 h at 37° C. or 39.5° C.

Codon usage for each of the mutations was as follows: L342A-GCG; D349R-CGT; L446A GCT; R455D-GAC; R456D-GAC; L446A, R455D-GCT (Ala), GAC (Asp). The sequences for each mutant were confirmed by DNA sequencing. Two independent isolates of each mutant were generated.

For wild-type, L342A and D349R, 0.4 ug DNA per plate yielded 10-50 plaques, and 4 ug DNA per plate destroyed the monolayer. For L446A, R455D, R456D and L446A:R455D, neither DNA concentration yielded detectable virus in duplicate transfections.

Single plaques of wild-type, L342A, and D349R viruses were isolated at 32.5° C and used to infect HeLa cells to prepare high-titer virus stocks. Phenotypes of high-titer stocks were identical to those of the original plaque isolates, as determined by plaque assay preformed as described previously (Kirkegaard K. J Virol. 64:195-206 (1990)).

Analysis of the in vivo phenotype of each of the Interface I mutants yielded results consistent with those observed for both RNA binding and extension in vitro. In the in vitro binding and extension studies, the D349R and L342A Interface I mutants were generally the least affected, correlating well with the phenotypes observed in cells, where the D349R (WT phenotype) and the L342A (ts phenotype) were the only Interface I mutants to display any level of infectivity. In the in vivo assay, only the D349R mutant displayed an infectivity similar to the WT poliovirus. The L342A mutant displayed WT infectivity at 32.5° C., but displayed a small plaque phenotype at both 37° C. and 39.5° C. All of the other Interface I mutants did not display any viral infectivity at any of the temperatures tested. Thus, interfering with the formation of Interface I reduces RNA binding affinity and destroys viral infectivity.

### Example 5

Involvement of both Interface I and Interface II in Polymerase-Polymerase Binding

Evidence that both Interface I and Interface II are involved in the polymerase oligomerization is reinforced by the introduction of point mutations in poliovirus polymerase. The protein-protein interactions between poliovirus polymerase molecules are difficult to disrupt by single point mutations. Point mutations that disrupt poliovirus 3D-3D interactions have been screened in the two-hybrid system. None has been identified, even though parallel screens to look for mutations that disrupt interactions between 3D and other protein ligands have been very successful (Hope, D. A et al.., J. Virol. 71:9490-8 (1997)). This results, at least in part, from the 3D-3D interactions being very robust in the two-hybrid system, in which multiple lex-3D fusion proteins bind to the promoter that controls β-galactosidase synthesis, and it is difficult for individual point mutations to destroy this observed interaction. Inspection of the two interfaces in FIG. 2 suggests both interfaces must be disrupted to eliminate the signal in the two-hybrid system.

### Example 6

Mutational Analysis of Interface II and the N-terminal regions of Poliovirus Polymerase

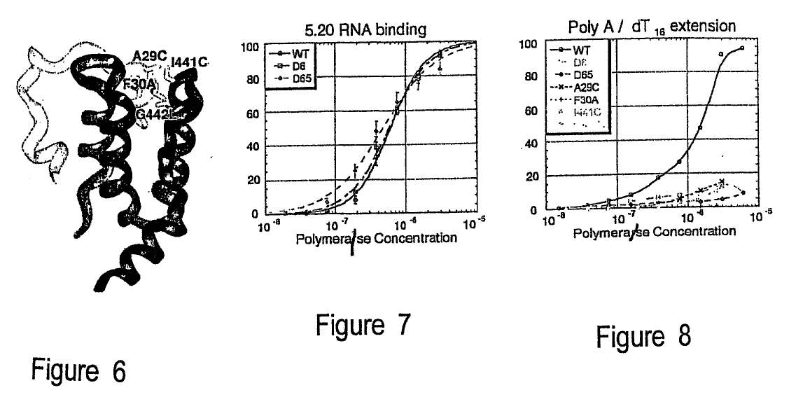

Since the N-terminal residues of poliovirus polymerase are critical for activity (Plotch S. J. et al. J Virol. 63:216-25 (1989)) and because the N-terminal most polypeptide segment is intimately associated with the top of the thumb subdomain at Interface II (FIG. 2C), this unusual interaction was explored. Several different point mutations in the N-terminal strand (A29C, F30A) and in the top of the thumb subdomain (G442L, I441C) (FIG. 6) as well as deletions of the first six (D6) and 65 (D65) N-terminal amino acids of poliovirus polymerase were constructed.

Interestingly, RNA binding by the N-terminal deletion mutants (FIG. 7) using the 5.20 RNA was indistinguishable from the WT enzyme. Even deleting the first 65 residues, which eliminates the N-terminal strand and, therefore, a significant portion of Interface II, does not affect RNA substrate affinity.

In contrast, the N-terminal deletion mutants and the point mutants in the N-terminal interaction region were all unable to incorporate a significant amount [α-³²P] UTP into dT₁₆-primed poly-A substrate, although trace levels of incorporation were observed for the point mutants and the D6 deletion mutant at the highest polymerase concentration tested (FIG. 8). The result that mutation of residues involved in the N-terminal strand interactions and, therefore, Interface II dramatically reduce enzymatic activity but do not significantly affect RNA binding contrasts with the results of mutations at Interface I, which affect RNA binding but retain significant catalytic activity.

In fact, when truncated versions of poliovirus 3D, in which both the bait and the prey contained only residues 1-136, and the prey contained residues 65-461 a substantial interaction signal was still observed. This interaction signal was sufficiently strong that yeast colonies containing these plasmids were medium-to-dark blue when grown on X-gal-containing plates. Since the N-terminal residues should be capable of interacting across Interface II of the prey plasmid if properly folded, but should be unable to form Interface I, LexA-PV3D (1-136) bait and the B52-PV3D (65-461) prey bind due to the interaction across Interface II. Furthermore this interaction is sensitive to mutations AL 333 and AL34, both predicted to disrupt Interface I, but not mutation AL 28, predicted to disrupt Interface I. Therefore, the two-hybrid screen can be used to assay for the formation of Interface II and hence its disruption.

The V33A/F34A mutations, shown to abrogate the formation of interface II in the two-hybrid system, also led to a loss of viral viability when reconstructed into an infectious poliovirus cDNA. this loss of viral viability is consistent with a requirement for polymerase-polymerase interactions across interface II for polymerase function in vivo.

### Example 7

Disulfide Crosslinking Analysis of the N-terminal Strand Interaction

Interface II involves two different ordered portions of the N-terminal polypeptide regions of poliovirus polymerase. Residues 13-35 form a strand, which begins in the active site cleft of the polymerase and loops over the top of the thumb subdomain. Three large hydrophobic residues in this strand, Phe30, Val33, and Phe34, appear to anchor this strand to the top of the thumb subdomain by wedging into the hydrophobic core of the thumb subdomain (See FIG. 2). Residues 69-97 form an α-helix that is positioned at the bottom of the fingers subdomain. Unfortunately, the residues connecting these two ordered portions of the N-terminal polypeptide regions (residues 36-68) are disordered in the crystals. Although this is possible, it is also possible that these residues might connect by an intermolecular interaction via Interface II, a distance of <32 Å (FIG. 6). Thus the very N-terminus of poliovirus polymerase, which is critical for catalytic activity is contributed in traps by an adjacent polymerase molecule associated via Interface II.

In order to determine whether residues 13-35 derive from an inter- or an intramolecular interaction, a disulfide crosslinking experiment was performed. Three separate polymerase mutants were constructed, one which introduced a Cys residue in the N-terminal strand (A29C), a second mutant which introduced a Cys residue at the top of the thumb subdomain (I1441C), and a third which introduced both of these Cys mutations (A29C:I441C). These positions, A29 and 1441, are positioned directly across from each other at Interface II, such that the predicted positions of the sulfur atoms would be <4.0 Å apart.

In the crosslinking experiment, samples containing WT and mutant polymerases were incubated individually and together in the absence of reducing under quite mild oxidizing conditions, i.e. no oxidizing agents were intentionally added, and the samples were simply incubated for 8 hours in the absence of DTT. Several diffuse bands with greater than dimer mobilities were observed for the individual mutants as well as the mixtures with the WT enzyme, but this is not surprising since there are five other naturally occurring cysteine residues in poliovirus polymerase which would provide ample opportunity for formation of disulfide bonds. However, the mixture of the two individual Cys mutants yielded a discrete dimer length product consistent with the formation of a disulfide crosslink between the A29C and I441C mutant polymerases. The amount of this dimer sized product is substantial (approximately 15% of the initial material) considering that the yield is likely limited by formation of other disulfide products and incomplete reaction, indicating that in solution a substantial portion of the polymerase associates via an intermolecular manner at Interface II. Samples in which 50 mM DTT was added just prior to the crosslinking reaction display only monomer length products, and thus the “non-monomeric” products observed in the samples that were not treated by DTT are were due to disulfide crosslinking and not some other crosslinking reaction.

Crosslinking data for the A29C:I441C doubly mutant polymerase showed a dimer length product of the same molecular weight as the mixture of the A29C and I441C individual mutant polymerases. However, the yield of the dimeric product was lower and additional higher molecular weight products were also observed higher up on the gel and in the well for the A29C:I441C double mutant polymerase. These larger products likely arise from formation of trimeric, tetrameric, etc. disulfide crosslinked products that became possible with the A29C: I441C double mutant. Note that because of the more extensive crosslinking for the double mutant, the reactions were incubated for 6 rather than 8 hours.

The N-terminus of the poliovirus polymerase is known to be important for polymerase activity in that deletion of a Trp residue at position 5 of the polymerase results in a loss of replication activity (Plotch S. J. et al J Virol. 63:216-25 (1989)). In the structure of poliovirus polymerase, the N-terminus is bound in the active site cleft of the polymerase. In order to better understand the role of the N-terminus of poliovirus polymerase as well as the interaction at Interface II, two different deletion mutants were constructed, which remove the first 6 (D6) or 65 (D65) N-terminal amino acids, and also a number of point mutations (A29C, F30A, I441C and G442L) which should disrupt the interaction between the N-terminal strand and the top of the thumb subdomain at Interface II.

These N-terminal mutant polymerases, in contrast to the Interface I mutant polymerases, exhibited solubility properties very similar to that of the WT polymerase. These studies also showed that the N-terminal mutants bound the 5.20 RNA with a Kd essentially identical to that of the WT. However, each of the N-terminal mutant polymerases was dramatically reduced in catalytic activity. These data show that, unlike Interface I, which appears to be involved in RNA binding but not catalysis, the N-terminal strand and its interaction at Interface II are important for catalysis but not RNA binding.

A disulfide crosslinking experiment in which Cys residues were introduced into the top of the thumb subdomain (I441C) and into the N-terminal strand (A29C). Mixing these two mutant polymerases together should, upon oxidation, yield a discrete dimeric product if the N-terminal strand association is in trans. This dimeric product was indeed observed, indicating that the N-terminal strand is contributed in trans from another polymerase molecule. The disulfide crosslinking reactions contained 7.5 μM total protein, 50 mM Pipes pH 6.6, I mM ATP and 0.35 mM DTT (50 mM DTT for reduced samples). The reaction was initiated by mixing the protein with the DTT and allowing this to sit on ice for 15 min. The Pipes and ATP were then added such that the final volume was 50 pM. About one-sixth of the reaction volume was polymerase such that the reaction also contained 80 mM NaCI. The reaction was then incubated at 30° C. for 8 hrs. The reaction was quenched by adding 21 μL gel loading buffer (50 mM Tris pH 6.8, 4.4% SDS and 8% glycerol). The samples were then boiled for two minutes and then loaded onto a 10% polyacrylamide gel. The gel was then visualized through Coomassie staining.

Analysis was also carried out with an A29C:I441C doubly mutant polymerase to evaluate for intra-molecular disulfide bond formation, but the results of this experiment were complicated by the fact that each doubly mutant polymerase could potentially form two disulfide bonds leading to formation of oligomeric structures. Importantly, no new crosslinked species were observed migrating faster than the reduced form of the polymerase or intermediate between the uncrosslinked and dimeric species as one might expect if the crosslinking also occurred on an intramolecular manner. Only dimer and larger sized products were observed, indicating that the N-terminal strand observed at Interface II derived from an intermolecular interaction and that interaction of polymerase molecules via Interface II is not limited to dimerization, but can yield more extended structures.

### Example 8

Dilution Studies with WT Polymerase

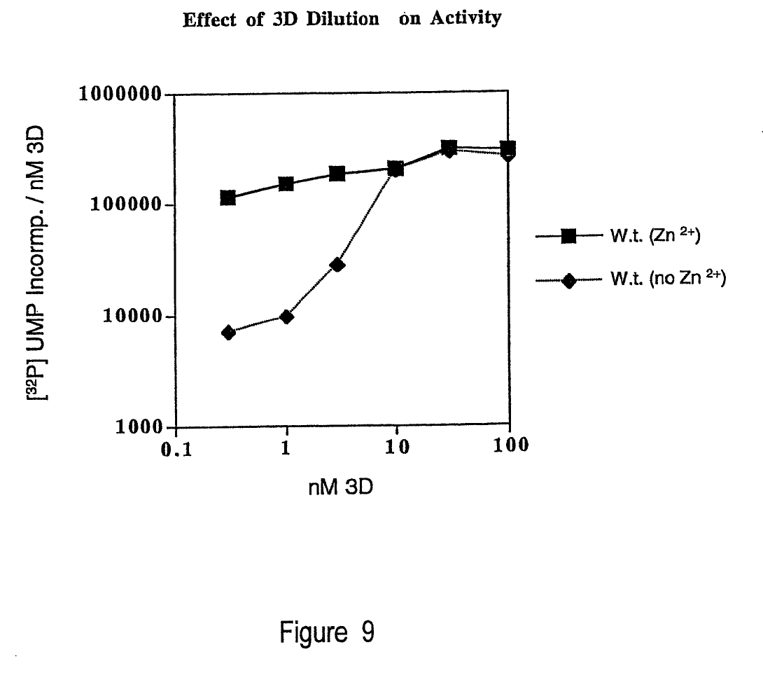

If poliovirus polymerase interactions occur in solution and are in some way limiting in enzymatic assays, one would expect specific activity to vary as a function of enzyme concentration. The specific activity of poliovirus polymerase was investigated at various concentrations using standard enzymatic assay conditions in which substrate is in excess and enzyme is limiting. In the absence of Zn²⁺, specific activity ([α-³²P] UMP incorporation/nM 3D) indeed decreased over 10 fold for a change in enzyme concentration from 10 nM to 1 polymerase (FIG. 9). However, above polymerase concentrations of 10 nM, or at all concentrations tested in the presence of Zn²⁺, the specific activity varied less than 3-fold over the entire concentration range tested. These results indicate that at concentrations above 10 nM or in the presence of Zn²⁺, polymerase-polymerase interactions are not limiting for polymerase activity. In the absence of Zn²⁺ and at concentrations of less than 10 nM, however, specific activity indeed changes with changing enzyme concentration.

An important extension of previous studies of poliovirus polymerase activity in solution is the analysis of specific activity as a function of polymerase concentration described here. If polymerase-polymerase interactions are important then one would predict that in conditions where these interactions are limiting (i.e. oligomeric structures do not pre-form prior to a subsequent rate limiting step), specific activity would decrease with decreasing polymerase concentration. Experiments show that indeed the specific activity decreases over 10 fold when poliovirus polymerase concentration is decreased from 10 to 1 nM. At polymerase concentrations >10 nM, specific activity is no longer concentration dependent, indicating that the active and/or oligomeric form is stable and no longer limiting in the experiment.

Interestingly, including 60 μM ZnCl₂ in these assays nearly eliminates the decrease in specific activity with decreasing polymerase concentration. These data provide an explanation for the well known observation that in standard assay conditions, Zn²⁺ stimulates the activity of poliovirus polymerase in solution.

Possible interpretations are that protein-protein interactions are facilitated by ZnCl₂ or that the active conformation is stabilized by the presence of this metal by some other means. Interestingly, a Ca²⁺, metal binding site is observed in the crystal structure at Interface II (FIG. 2B). Perhaps Zn²⁺ could coordinate in this same site and stabilize interactions at Interface II such that this interaction would no longer be limiting in the assay. Zn²⁺, in fact, does facilitate aggregation of poliovirus polymerase molecules in solution in that at polymerase concentrations >1 μM the polymerase becomes very insoluble in the presence of Zn²⁺. Zn²⁺ is known to coordinate and stabilize ordered protein-protein assemblies as has previously been observed with tubulin, where its presence induces formation of ordered tubulin “sheets” (Nogales E. et al., Cell 96:79-88 (1999); Downing K. H. and Nogales E., Curr Opin Cell Biol. 10:16-22 (1998)).

Enzymatic elongation assays for the wild type and mutant polymerases were carried out in: 50 mM HEPES, pH 7.2; 0.5 mM of each of ATP, GTP, and CTP; 50 pM UTP (for assays containing 0.3 nM to 10 nM 3D) or 500 μM UTP (for additional assays containing 10 nM to 100 nM 3D); 4 μCi/incubation (30 μL NEN, 3000 Ci/mmol); 4 mM DTT; 3 mM magnesium acetate; 0.1% NP40; μpoly(A) and 20 μM oligo(U) (for assays containing 0.3 to 10 nM 3D) or 3 μM poly(A) and 60 μM oligo(U) (for assays containing 10 nM to 100 nM 3D); 60 pM ZnCl₂ (where indicated); and 3D (0.3 to 100 nM; dilutions in 50 mM HEPES, pH 7.2, 0.1% NP40, 50 mM KCI, and 5 mM β-mercaptoethanol). 3D concentrations were determined as described above. Reaction mixes were prepared at 0° C. and the mixes were pre-incubated at 0° C. for 60 min. Incubations were then at 30° C. for 10 min in order to obtain initial linear rates and were performed in duplicate for any given set of assays. Assays were repeated 2 to 3 times for each 3D preparation. Aliquots were removed at 0 min and 10 min and assayed for acid precipitable material. In order to normalize assays from day to day and from one 3D preparation to another, total cts/min were determined for each reaction mix and normalized to a common value. Zero time cts/min were subtracted from 10 min values to obtain net cts/min for each assay. Finally specific activities were plotted to illustrate any deviation from a constant specific activity.

Elongation of dT₁₆ primer in the presence of poly-A template was evaluated by measuring incorporation of [α³²P] UTP into RNA products. Various concentrations of polymerase from 7.5 nM to 3.0 pM were incubated with 2.5 ug/mL poly-A, 46 nM dT₁₆ and elongation buffer (25 mM Mes pH 6.5, 2.5 mM Mg(OAC)₂, 50 mM NaCl, 2 mM DTT, 50 pM UTP, 10% glycerol and 0.01 μCi/μL [α³²P] UTP) for 30 min. at 30° C.Incorporation of the radiolabel was evaluated by filtering 27 μL of the reaction through Hybond N+ nylon membrane in a 96 well filter apparatus. Each well was washed with a total of 3 mL of 5% w/v sodium phosphate, 2% w/v sodium pyrophosphate to remove unincorporated label. The amount of ³²P that remained bound to the Hybond paper was quantitated by phosphorimager analysis.

Elongation assays of the 5.20 RNA were performed as follows. The polymerase was incubated with 25 mM Mes pH 6.5, 2.5 mM Mg(OAc)₂, 25 mM NaCl, 2.0 mM DTT, 10% glycerol, 40 pM ZnS0₄, 0.5 mM of each NTP and 10 nM [α-³²P] labeled 5.20 RNA at 30° C. for 15 min. After the incubation, the samples were placed on ice and 1/10 volume (2 μL of 50 mM Tris 7.5, 20 mM EDTA and 2% SDS was added to each sample. Elongated products were then separated by electrophoresis on a 20% native polyacrylamide (19:1)/1.0×TBE gel. The amounts of elongated and unelongated products were measured using a phosphorimager (Molecular Dynamics).

### Example 9

Identification of Polymerase Interactions at Interface I and at Interface II Using a Yeast Two-Hybrid Method

A yeast two-hybrid system was used to test the homologous and heterologous interactions of a number of viral RNA-dependent RNA polymerase. The system used identifies protein interaction between a “bait” hybrid protein, which contains the DNA-binding domain of bacterial repressor protein LexA, and a “prey” hybrid protein, which contains sequences that can activate transcription by reconstituting a transcription factor sufficient to activate transcription from the plasmid-encoded reporter gene, β-galactosidase, that is downstream of the lexA recognition sequences for LexA (Gyuris, J. et al. Cell 75: 791-803 (1993)). To detect interaction between the bait and prey hybrid proteins, however, it is crucial that neither hybrid protein can activate transcription of the reporter gene on its own.

To test interactions between homologous and heterologous polymerases, coding regions from the polymerases of poliovirus Mahoney type 1, human rhinovirus 14, enterovirus 71, Sindbis virus, yellow fever virus and Dengue virus were used in both bait and prey vectors. The cDNAs for each were cloned into the bait and prey vectors, in-frame with the upstream lexA or transcriptional activation domain sequences, respectively, to which the polymerase were fused. PCR amplification was performed for fewer than 10 cycles and all sequences amplified by PCR sequenced to ensure that nucleotide changes are not introduced by amplification.

In all, 5 bait clones and 4 prey clones were used, as shown below in Table 1. For each bait and prey construct, controls were performed to ensure that the fusion protein encoded by that plasmid, alone, does not activate transcription of the reporter gene. In all, 49 initial strains will be tested for activation of the β-galactosidase gene. Each of these strains had yeast strain EGY40 as a parent and contain the β-galactosidase reporter gene. Every combination of bait and prey was tested using a simple color assay on plates. In the presence of the β-galactosidase substrate X-gal, colonies that express β-galactosidase are blue, and even very light blue colonies are easy to identify. The level of β-galactosidase activation was quantified for each pairing, and is expressed by +'s as follows:

TABLE 1
Activation of β-galactosidase using bait and prey polymerase pairs

| | | | | | control |
|---|---|---|---|---|---|
| | Polio | HRV14 | HAV | Sindbis | vector |
| Polio | +++ | +++ | + | ++ | − |
| | (591 ± 71) | (963 ± 272) | | | |
| HRV14 | +++ | +++ | + | ++ | − |
| | (687 ± 15) | (632 ± 127) | | | |
| Ent71 | +/− | + | + | + | − |
| | (23 ± 2) | (65 ± 4) | | | |
| HAV | − | − | − | + | − |
| Dengue | − | − | + | − | − |
| Vector | − | +/− | − | +/− | − |
| | (9 ± 2) | (35 ± 4) | | | |

Two-hybrid signal for different combinations of “bait” and “prey” polymerases is shown qualitatively, for the intensity of blue color on plates containing X-gal (+++, dark blue; ++ medium blue; + light blue,− white). For some combinations, the amount of beta-galactosidase activity made following growth in liquid medium has been quantified; these activities are given in Miller units below the qualitative signal. Clearly, both homologous and heterologous interactions can be observed in the two-hybrid system.

The interactions of heterologous and homologous RNA-dependent RNA polymerases were further examined in the yeast two-hybrid system. Yeast cultures were transformed with bait(Lex; Lex202) and prey(pJG; pJG4.5) plasmids containing the RNA-dependent RNA polymerase coding regions from poliovirus 3D, rhinovirus 14 3D, enterovirus 71 3D, hepatitis A 3D or Sindbis virus nsP4. The expression of all proteins was confirmed by immunoblotting. To assay for interactions between the respective bait and prey viral polymerase fusion proteins, β-galacotsidase activity was measured using the Miller permeabilized-cell assay. The amount of beta-galactosidase activity observed for each combination of bait and prey is expressed relative to the activity observed for the poliovirus 3D-poliovirus 3D interaction (LexPolio3D-pJGPolio3D) under the same reaction conditions. The results are shown in FIG. 14. These results again confirm that both homologous and heterologous interactions can be observed in the two-hybrid system

To examine the formation of Interface II interactions between heterologous polymerases, bait and prey combinations were constructed to test whether the N-terminal sequences of polymerases from rhinovirus 14 and rhinovirus 16 could interact directly with poliovirus polymerase in the yeast two-hybrid system. This combination of bait and prey precluded the possibility of interaction at Interface I, allowing only Interface II interactions. Yeast cultures were transformed with the bait Polio 3D d65 plasmid (residues 66-461 of poliovirus polymerase) and the prey HRV(1-136) plasmid (residues 1-136 of either rhinovirus 2 (HRV2), rhinovirus 14 (HRV 14) or rhinovirus 16 (HRV16)). To assay for interactions between the respective bait and prey viral polymerase fusion proteins, β-galacotsidase activity was measured in Miller units. The results (FIG. 15) show clearly that heterologous interactions can occur across Interface I. Similar experiments (not shown) argue that heterologous interactions are made across Interface II as well.

### Example 10

Identification of a Peptide Pharmacophore to Interface I

A peptide inhibitor of poliovirus/poliovirus interactions has been designed based on an alpha-helical region at Interface I, and shown to inhibit poliovirus polymerase activity in vitro by reducing the polymerase's ability to oligomerize into two-dimensional lattices and to bind to substrate RNA with high affinity.

To solubilize the alpha-helical peptides derived from Interface I, a peptide was synthesized that was predicted to fold into a canonical “zinc finger”, in which two beta sheets coordinated by a zinc ion hold an alpha helix in a defined orientation with the “outer” residues of the alpha helix were engineered to mimic the alpha helix present at Interface I of poliovirus polymerase. Peptide 1 is soluble in aqueous buffers, and inhibits poliovirus polymerase effectively.

A synthetic peptide was designed to mimic the continuous α-helix at Interface I in the context of a Zinc-finger motif to improve solubility. A search of the Gen Bank database revealed a sequence from 2GLI, (Pavletich and Pabo, Science 216:1701(1993)) a human transcription factor, that contained an α-helical region similar to that from Interace I; the entire motif sequence was (KPHKCTFEGCRKSYSRLENLKTHLRSH)(SEQ ID NO:4). Five changes to the sequence were made so that the peptide, Peptide 1, would even more closely resemble the α-helix at Interface I. The peptide sequence, KPHKCTFEGCRKSYSRSTNLRRHLNSH (SEQ ID NO:5) was synthesized by Sigmagenosis.

To disassemble the preformed polymerase oligomers, both polymerase and peptide were treated with 15% acetonitrate, after which the acetonitrate was removed by dialysis.

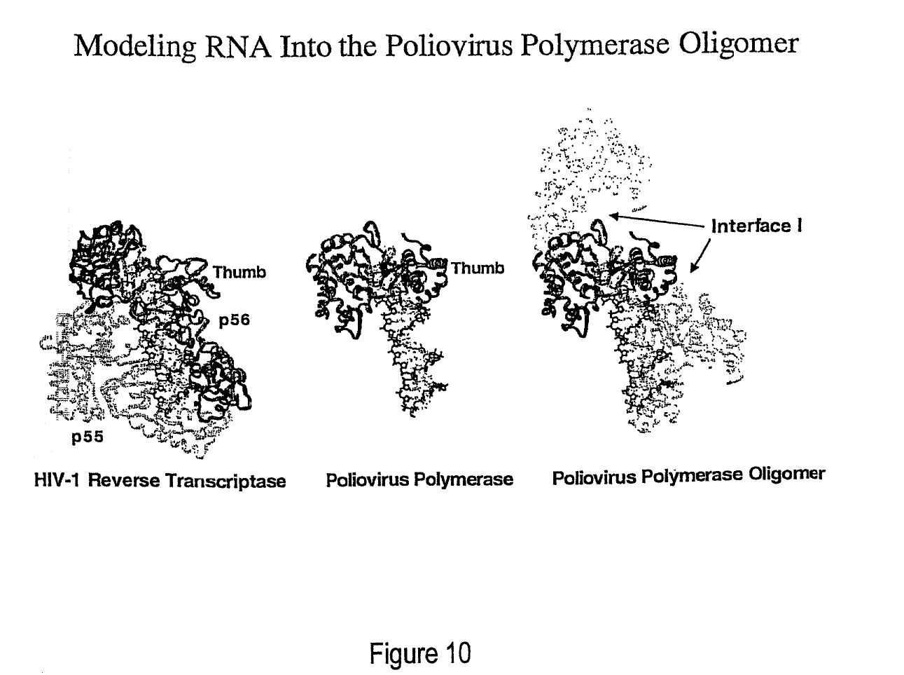

The elongation of a radiolabeled, self-priming RNA, present at approximately 10 nM, was monitored as a function of polymerase concentration. Polymerase was denatured in the presence of 15% acetonitrile either with no peptide, 10 μM peptide or 25 μM peptide. The acetonitrile and free peptide was removed by dialysis. The presence of the peptide causes a loss of cooperativity of the elongation reaction at both concentrations, and marked inhibition of the polymerase reaction at the higher concentration. (FIG. 10).

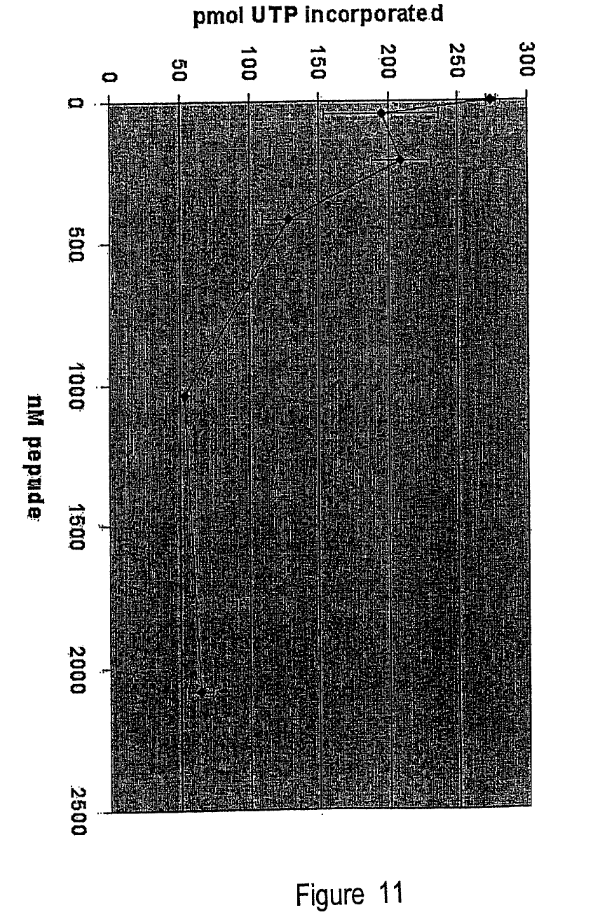

Polymerase activity was also determined on homopolymeric RNAs at high concentrations but in the presence of limiting primer. The incorporation of α-³²P-labeled UTP into polynucleotides was measured by DEAE filter binding. Poly(A) was used as a template at a concentration of 4 μM (in nts), with the oligo dT primer present 1 μM (in nts). When the concentration of peptide in the denaturation reaction was increased, the amount of UTP incorporated diminished (FIG. 11).

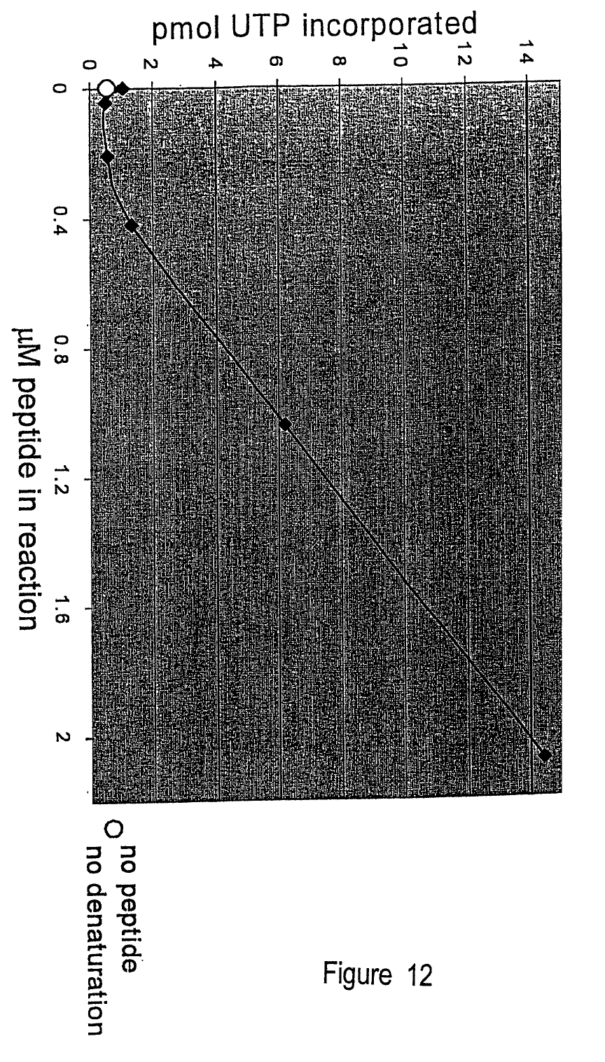

Polymerase activity was then tested on homopolymeric RNA templates in the presence of high concentrations of primer. Under these conditions of high template and primer concentrations, neither RNA binding nor polymerase processivity should be rate-limiting, therefore no effect of disruption of Interface I is expected or seen. The incorporation of α-³²P-labeled UTP into polynucleotides was measured by DEAE filter binding. Both poly(A) and oligo d(T) were present at 4 μM, in nucleotides; polymerase concentration was 200 nM. The dramatic stimulation observed in the presence of peptide (FIG. 12) was likely to be the result of the disruption of large polymerase complexes, effectively increasing the concentration of polymerase active sites.

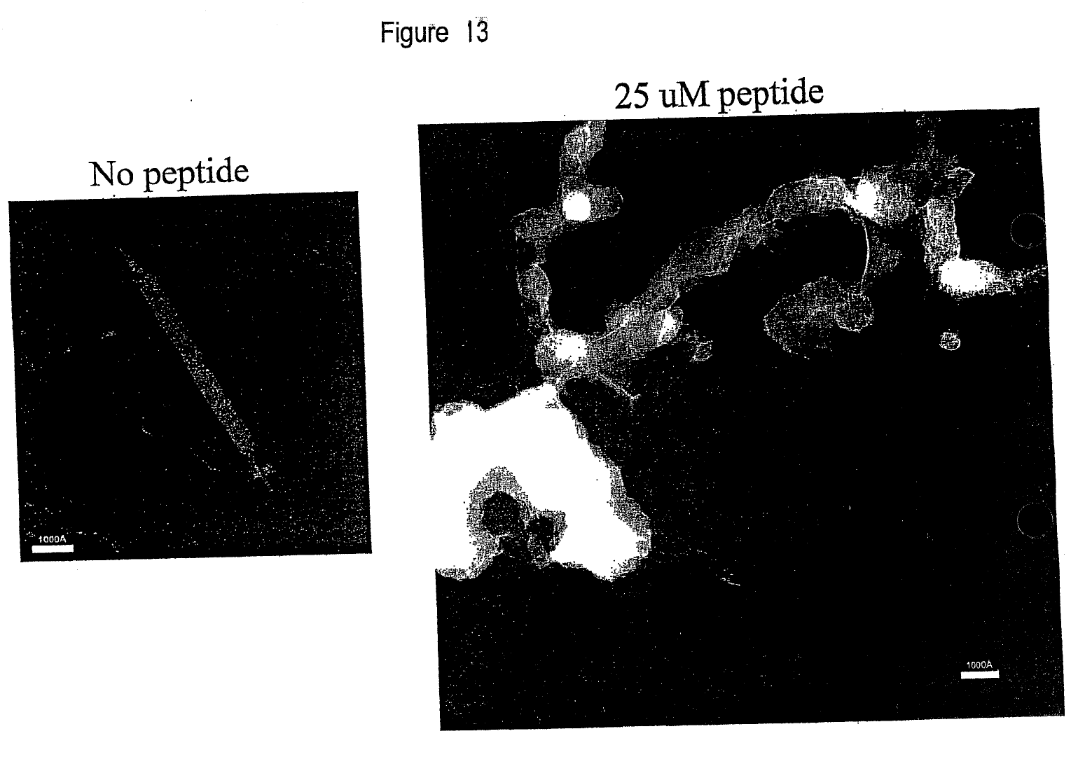

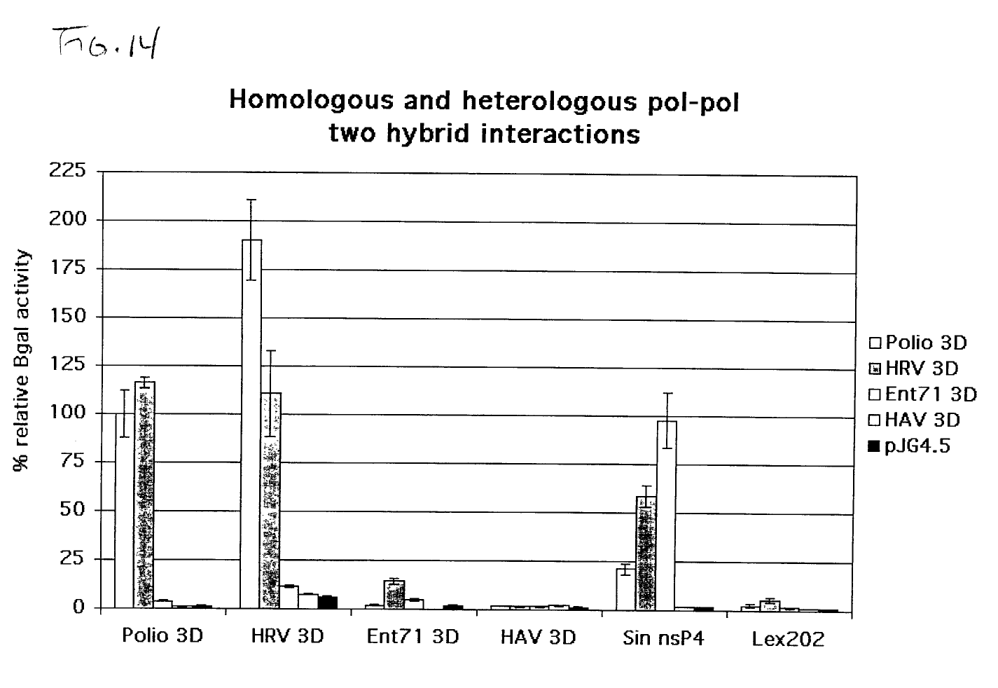

The ability of Peptide I to disrupt the lattices of poliovirus polymerase was demonstrated by electron microscopy visualization. Sheets and tubes of polymerase observed by electron microscopy in the absence of peptide (A) are replaced by large aggregates and more narrow ribbons when Interface I is disrupted by the AL28 mutations (not shown) or the presence of peptide (B) (FIG. 13).

The peptide inhibitor of poliovirus inhibitor also inhibits the activity of the rhinovirus polymerase, thus showing that this protein-protein interface is functionally and structurally conserved.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A pharmacophore of a binding surface of a viral RNA-dependent RNA polymerase, wherein the pharmacophore is characterized by a) selective binding to a binding surface on a viral RNA-dependent RNA polymerase and b) inhibition of viral RNA-dependent RNA polymerase activity.

2. The pharmacophore of claim 1, wherein the pharmacophore inhibits interaction of polymerase-polymerase binding by interaction with Interface I of a viral RNA-dependent RNA polymerase.

3. The pharmacophore of claim 2, wherein the pharmacophore inhibits polymerase-polymerase binding by selectively binding to a surface structurally defined by poliovirus RNA-dependent RNA polymerase residues 342 and 349 or corresponding positions thereof of a RNA-dependent RNA polymerase.

4. The pharmacophore of claim 2, wherein the pharmacophore binds selectively to an binding surface structurally defined by poliovirus RNA-dependent RNA polymerase residues 446, 455 and 456 or corresponding positions thereof of a RNA-dependent RNA polymerase.

5. The pharmacophore of claim 1, wherein the pharmacophore inhibits interaction of polymerase-polymerase binding by interaction with Interface II of a viral RNA-dependent RNA polymerase.

6. The pharmacophore of claim 5, wherein the pharmacophore inhibits polymerase-polymerase binding by selectively binding to a surface structurally defined by poliovirus RNA-dependent RNA polymerase residues 30, 33 and 34 or corresponding residue positions thereof of a RNA-dependent RNA polymerase.

7. The pharmacophore of claim 1, wherein the polymerase is a picornaviral RNA-dependent RNA polymerase.

8. The pharmacophore of claim 1, wherein the pharmacophore comprises a peptide.

9. The pharmacophore of claim 8, wherein the peptide further comprises an element that facilitates entry into a host cell.

10. The pharmacophore of claim 8, wherein the peptide comprises the sequence of SEQ ID NO:5.

11. The pharmacophore of claim 1, wherein the pharmacophore is an antibody immunospecific for a polymerase-polymerase binding surface of a viral RNA-dependent RNA polymerase.

12. The pharmacophore of claim 1, wherein the pharmacophore is a small molecule.

13. The pharmacophore of claim 1, wherein the pharmacophore is detectably labeled.

14. A composition for treating a viral infection, comprising:
a pharmacophore characterized by a) selective binding to a binding surface of a viral RNA-dependent RNA polymerase and b) activity in disruption of viral RNA-dependent RNA polymerase activity; and
a pharmaceutically acceptable carrier.

15. The composition of claim 14, wherein the pharmacophore is further characterized by activity in disruption of a plurality of positive strand virus.

16. The composition of claim 14, wherein the pharmacophore has activity in disruption of a picornavirus RNA-dependent RNA polymerase.

17. A method of treating viral infection in a subject, comprising the step of administering to the subject a composition of claim 14.

18. The method of claim 17, wherein the subject is mammalian.

19. A computer comprising a representation of a pharmacophore in computer memory that either designs a molecular structure that possesses a biological activity or screens a molecular structure for possession of the biological activity wherein the pharmacophore comprises:
a three-dimensional array of points defining a specific shape and volume, wherein the three-dimensional array of points is an aggregate average shape of a molecule or a plurality of molecules that optimally fit a binding interface of a viral RNA-dependent RNA polymerase, wherein the aggregate average shape is represented by a coordinate system configured in computer memory, and the molecule or the plurality of molecules possess the same or similar biological activity.

20. The computer of claim 19, wherein the pharmacophore binds Interface I of a viral RNA-dependent RNA polymerase.

21. The computer of claim 20, wherein the pharmacophore selectively binds to a surface defined by poliovirus RNA-dependent RNA polymerase residues 342 and 349 or corresponding positions thereof of a RNA-dependent RNA polymerase.

22. The computer of claim 20, wherein the pharmacophore binds selectively to an binding surface structurally defined by poliovirus RNA-dependent RNA polymerase residues 446, 455 and 456 or corresponding positions thereof of a RNA-dependent RNA polymerase.

23. The computer of claim 19, wherein the pharmacophore inhibits interaction of polymerase-polymerase binding by interaction with Interface II of a viral RNA-dependnet RNA polymerase.

24. The pharmacophore of claim 23, wherein the pharmacophore inhibits polymerase-polymerase binding by selectively binding to a surface structurally defined by poliovirus RNA-dependent RNA polymerase residues 30, 33 and 34 or corresponding residue positions thereof of a RNA-dependent RNA polymerase.


SEQUENCE LISTING (5 sequences)

SEQ ID NO: 1 - DNA, 48 bp, Artificial Sequence
  template DNA for T7 transcription reaction
ggcagctgcatgcagctgccgcgcctatagtgagtcgtattaatttcg

SEQ ID NO: 2 - DNA, 43 bp, Artificial Sequence
  template DNA for T7 transcription reaction
ggcagctgcatgcagctgcctatagtgagtcgtattaatttcg

SEQ ID NO: 3 - DNA, 24 bp, Artificial Sequence
  template DNA for T7 transcription reaction
cgaaattaatacgactcactatat

SEQ ID NO: 4 - protein, 27 aa, Homo sapiens
KPHKCTFEGCRKSYSRLENLKTHLRSH

SEQ ID NO: 5 - protein, 27 aa, Artificial Sequence
  synthetic peptide
KPHKCTFEGCRKSYSRSTNLRRHLNSH